(19) United States
(12) Patent Application Publication
(10) Pub. No.: US 2013/0217672 A1
(43) Pub. Date: Aug. 22, 2013

(54) (+)-3-HYDROXYMORPHINAN-BASED POLYCYCLE DERIVATIVES

(71) Applicants: Jinhwa Lee, Yongin-si (KR); Jong Yup Kim, Yongin-si (KR); Jeongmin Kim, Yongin-si (KR); Kwang Woo Ahn, Yongin-si (KR)

(72) Inventors: Jinhwa Lee, Yongin-si (KR); Jong Yup Kim, Yongin-si (KR); Jeongmin Kim, Yongin-si (KR); Kwang Woo Ahn, Yongin-si (KR)

(73) Assignee: GREEN CROSS CORPORATION, Yongin-si, Gyeonggi-do (KR)

(21) Appl. No.: 13/696,246

(22) PCT Filed: May 13, 2011
(86) PCT No.: PCT/KR2011/003548

§ 371 Date: Nov. 21, 2012

Related U.S. Application Data
(60) Provisional application No. 61/334,371, filed on May 13, 2010.

(51) Int. Cl. C07D 498/08; C07D 471/08; C07D 491/08

(52) U.S. Cl. CPC C07D 498/08; C07D 491/08; C07D 471/08
USPC 514/211.1; 514/229.5; 544/99; 540/546; 514/281; 546/43; 514/250; 544/342; 544/338; 546/40; 514/279

(57) ABSTRACT

A (+)-3-hydroxymorphinan-based polycycle derivative of formula (I) is effective as a neuroprotective agent for neurodegenerative diseases including Alzheimer's disease, Parkinson's disease, Huntington's disease, amyotrophic lateral sclerosis, and ischemic stroke.

### FIELD OF THE INVENTION

The present invention relates to novel (+)-3-hydroxymorphinan ((+)-3-HM)-based polycycle derivatives which are effective as neuroprotectants.

### BACKGROUND OF THE INVENTION

The concept of neuroprotection was applied to chronic diseases of the brain as well as acute neurological conditions, since some of the basic mechanisms of damage to the central nervous system (CNS) are similar in these conditions. Neurodegenerative disorders include Parkinson's disease (PD), Alzheimer's disease (AD), Huntington's disease (HD) and amyotrophic lateral sclerosis (ALS). Neuroprotection has been regarded to be the mechanism of action of some of the drugs used in the treatment of these conditions.

Neurodegeneration in PD, AD, and other neurodegenerative diseases seems to be multifactorial, in that a complex set of toxic reactions including inflammation, glutamatergic neurotoxicity, increases in iron and nitric oxide, depletion of endogenous antioxidants, reduced expression of trophic factors, dysfunction of the ubiquitin-proteasome system, and expression of proapoptotic proteins leads to the death of neurons. Gangliosides are the major class of glycoconjugates on neurons and carry the majority of the sialic acid within the CNS. Ganglioside synthesis is essential for the development of a stable CNS. Interruption of ganglioside synthesis produces CNS degeneration and modified axon-glial interactions [Yamashita, T. et al., PNAS, 2005, 102, 2725-2730]. Thus, the fundamental objective in neurodegeneration and neuroprotection research is to determine which of these factors embodies the primary event, the sequence in which these events occur, and whether they act in concurrence in the pathogenic process. This has resulted in the concept that drugs addressed against a single target will be ineffective and instead a single drug with multiple pharmacological properties or a cocktail of drugs may be more appropriate. Among the many factors involved, apoptosis and glutamate toxicity play an important role.

Apoptosis mediated by genetic programs intrinsic to the cell is being implicated in neurodegenerative disorders. During the normal development of the vertebrate nervous system, approximately 50% of the different types of neurons usually die right after they establish synaptic connections with their target cells. It has been hypothesized that this death is due to failure of these neurons to obtain adequate amounts of survival specific neurotrophic factors from target cells. The mechanism of death is postulated to be deprival of extracellular survival signals, which normally suppress apoptosis.

Many neurodegenerative disorders are distinguished by conformational alteration in proteins that result in misfolding, aggregation and intra- or extra-neuronal accumulation of amyloid fibrils. Molecular chaprones provide a first line of defence against misfolded, aggregation-prone proteins and are among the most potent suppressors of neurodegeneration known for animal models of human disease. A better understanding of the molecular basis of chaperon-mediated protection against neurodegeneration may result in the development of therapies for neurodegenerative disorders that are associated with protein misfolding and aggregation.

There are approximately 100 million people in the world and 800,000 people in the United States alone with Parkinson's disease (PD).

Parkinson's disease is a result of chronic progressive degeneration of neurons, the cause of which has not yet completely been clarified. While the primary cause of Parkinson disease is not known, it is characterized by degeneration of dopaminergic neurons of the substantia nigra (SN). The substantia nigra is a portion of the lower brain, or brain stem that helps control voluntary movements. The shortage of dopamine in the brain caused by the loss of these neurons is believed to cause the observable disease symptoms. Clinically, it manifests in the form of the cardinal symptoms resting tremors, rigor, bradykinesia, and postural instability.

Levodopa, dopamine agonists such as rotigotine, pramipexol, bromocriptine, ropinirol, cabergoline, pergolide, apomorphine and lisuride, anticholinergics, N-methyl D-aspartate (NMDA) antagonists, β-blocker as well as the monoamine oxidase-B (MAO-B) inhibitor selegiline and the COMT inhibitor entacapone are used as medicines for relief from the motor symptoms. Most of these agents intervene in the dopamine and/or choline signal cascade and thereby symptomatically influence the Parkinson-typical movement disorders.

In the present therapy for the Parkinson's disease, treatment is initiated after the appearance of the cardinal symptoms. In general, Parkinson's disease is said to be clinically evident if at least two of the four cardinal symptoms (bradykinesia, resting tremors, rigor, and postural instability) are detected and respond to L-dopa [Hughes, J Neurol Neurosurg Psychiatry, 1992, 55, 181]. Unfortunately, the motor function disorders in Parkinson patients become apparent only after about 70-80% of the dopaminergic neurons in the substantia nigra (SN) are irreparably damaged [Becker et al., J Neurol 249, 2002, Suppl 3:III, 40; Hornykiewicz, Encyclopaedia of Life Science 2001, 1]. Chances of a therapy with lasting effects are very bleak at that point. Hence, it is desirable to initiate the therapy as early as possible.

Current clinical observations as well as anatomical and genetic research show that diagnosis of Parkinson patients at an early stage and identification of high risk patients is possible. With that an opportunity arises for influencing the disease process at a point of time when more neurons are still there, rather than at the time of appearance of several cardinal motor symptoms of the Parkinson's disease, and thereby for protecting a quantitatively greater number of neurons. One can expect that the administration of an effective neuroprotective agent at an early stage will significantly delay the process of the development of the disease: The sooner the therapy is initiated, the higher are the chances of a long lasting prevention of the onset of symptoms, which degrade the quality of life.

Hence, such remedies are needed that not only influence the dopaminergic transmission and alleviate the symptoms of the Parkinson's disease in advanced stages, but also reverse, prevent, or at least significantly delay the dopaminergic neuron extinction in the early, to a great extent motor-asymptomatic, Parkinson stages [Dawson, Nat. Neurosci. Supplement, 5, 2002, 1058].

Alzheimer's disease (AD) is a progressive degenerative disorder of the brain that begins with memory impairment and eventually progresses to dementia, physical impairment, and death. Patients develop various psychiatric and neurological signs during the course of the disease. The prevalence rates of dementia vary significantly in different countries, but range from 2.1% to 10.5%. AD is the most common type of dementia. Several factors play a role in the etiology and pathogenesis of AD: aging; genetic risk factors; amyloid precursor protein and beta-amyloid accumulation; tau hyperphosphorylation; membrane disturbances, phospholipid metabolism, and disruption of signal transduction; inflammatory reactions and immunological disturbances; environmental toxins; neurotransmitter defects and imbalances; neuroendocrine disturbances; oxidative injury and free radicals, etc. AD is certainly not the result of a single operative mechanism but more likely comprises one or more processes that lead to intrinsic neuronal cell killing. A complex disease like AD is difficult to attack because no single approach is adequate and the development of a single universal therapy is unlikely. The most distinctive finding in the brains of patients with AD is copious deposits of amyloid β (Aβ). Aβ is found in small quantities in normal brains. Amyloid deposits by themselves do not damage the brain, but in the presence of apoE, amyloid forms into hair-shaped fibrils, and neuritic plaques [Holtzman, D. M. et al. PNAS, 2000, 97, 2892-2897]. The fact that apoE4 can increase both the amount of Aβ and the formation of amyloid fibrils seems to indicate that this version of the lipoprotein is a genetic risk factor for AD.

Current therapies involving cholinesterase inhibitors such as rivastigmine, donepezil and galantamine are not considered neuroprotective. These drugs act to increase brain acetylcholine and offset aspects of the cognitive decline during early stages of the disease. The efficacy of these compounds is modest and short-lived as the disease progresses. Since multiple mechanisms are involved in the pathogenesis of AD, current therapies target one of the several disturbances in AD. Free radical scavengers address at eliminating only one type of disturbance. One of the problems in designing reasonable therapies is dissent on the cellular events that elicit brain-cell death in AD and lead to dementia. One view is that amyloid plaques, composed mostly of the amyloid β protein, accumulate outside of brain neurons, growing larger and larger until they rupture the cells and kill them. Another view is that neurofibrillary tangles kill the cell. Some of the therapies related to neuroprotection include anti-inflammatory drugs, calcium channel blockers, antioxidants, glutamate antagonists, or inhibition of amyloid plaque formation.

The sirtuins are a family of enzymes which control diverse and virtual cellular functions, including metabolism and aging. Manipulations of sirtuin activities cause activation of anti-apoptotic, anti-inflammatory, anti-stress responses, and the modulation of an aggregation of proteins involved in neurodegenerative disorders. Recently, sirtuins were found to be disease-modifiers in various models of neurodegeneration. However, almost in all instances, the exact mechanisms of neuroprotection remain elusive. Nonetheless, the engineering of sirtuin activities is attractive as a novel therapeutic strategy for the treatment of currently neurodegenerative disorders such as AD and PD. There is a review article showing current data which support the putative therapeutic roles of sirtuin in aging and in neurodegenerative diseases and the feasibility of the development of sirtuin-based therapies [Kazantsev, A. et al. Biochim. Biophys. Acta, 2008, 1782, 363-369]. According to a literature, resveratrol, which is known to extend lifespan, improves mitochondrial function and protects against metabolic disease by activating SirT1 and PGC-1α [Lagouse, M. et al. Cell, 2006, 127, 1109-1122]. Another article reported that expression of SirT1 may be a good sensor of toxic neuronal processes, such as aging or neurodegenerative processes [Pallas, M. et al. Neurosci. 2008, 154, 1388-1397].

Ischemic stroke is a common life-threatening neurological disorder with severely limited therapeutic options. In the USA, stroke is the third leading cause of death and the major cause of disability. Care of the stroke survivors requires significant expense. More than 80% of stroke cases are ischemic, resulting from an obstruction of blood flow in a major cerebral artery by thrombi or emboli. Hemorrhagic stroke accounts for 15-20% of stroke cases. Currently, the only approved therapy for acute ischemic stroke is intravascular thrombolysis of the obstructing blood clot using recombinant tissue plasminogen activator (rTPA) such as Genentech's Altepase. Clearly there is an urgent need for a safe and efficacious neuroprotective agent. Despite progress in understanding the mechanism of ischemic damage, and identification of agents effective in animal models of stroke, clinical efficacy has not been achieved. Several drugs that have advanced into phase III clinical trials have been discontinued due to lack of efficacy or safety concerns.

(+)-3-HM and its derivatives have shown the neuroprotective property in 1-methyl-4-phenyl-1,2,3,6-tetrahydropyridine (MPTP) models for PD. In this animal model, daily injections with (+)-3-HM or its analogs showed that dopamine (DA) neurons in substantia nigra pars compacta have been protected and DA levels in striatum has been restored (US Patent Publication No. 2005-0256147 A1; International Patent Publication No. WO2005/110412; Zhang et al. FASEB J. 2006 Dec. 20(14):2496-2511; Zhang et al. FASEB J. 2005 Mar. 19(3):395-397; and Kim et al. Life Sci. 72 (2003) 1883-1895). However, (+)-3-HM and its derivatives are efficacious only if they are administered intraperitoneally or intravenously. The previous invention of our laboratories [Green Cross Corp., WO 2008/111767 (2008)] relates to an orally bioavailable, novel prodrug of (+)-3-HM which is effective as a neuroprotective agent for PD, when they are delivered orally. On the other hand, the present invention relates to provide novel (+)-3-HM-based polycycle derivatives which are effective as a neuroprotective pharmacotherapy for neurodegenerative diseases including AD, PD, Huntington's disease (HD), amyotrophic lateral sclerosis (ALS), and ischemic stroke.

### SUMMARY OF THE INVENTION

Accordingly, it is an object of the present invention to provide novel (+)-3-HM-based derivatives, which is effective as a neuroprotective agent for neurodegenerative diseases including Alzheimer's disease, Parkinson's disease, Huntington's disease, amyotrophic lateral sclerosis, and ischemic stroke.

It is another object of the present invention to provide a pharmaceutical composition for treating or preventing a neurodegenerative disease comprising the inventive compound as an active ingredient.

In accordance with one aspect of the present invention, there is provided a compound of formula (I), or a prodrug or a pharmaceutically acceptable salt thereof:

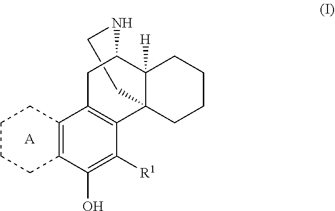

wherein, R¹ is H, C₁₋₃ alkyl, C₁₋₂ alkoxy, or halogen; and ring A is a saturated or unsaturated 5 to 9-membered heteromonocyclic ring, or a saturated or unsaturated fused 9 to 16-membered heterobicyclic ring, said heteromonocyclic and heterobicyclic rings each independently contains at least one heteroatom selected from N and O, and said ring A is optionally substituted by at least one substituent selected from the group consisting of halogen, hydroxy, C₁₋₄ alkyl, C₁₋₄ alkoxy, C₆₋₁₂ aryl, and C₃₋₁₁ heteroaryl.

In accordance with another aspect of the present invention, there is provided a pharmaceutical composition for treating or preventing a neurodegenerative disease, comprising the compound of formula (I) or a prodrug or a pharmaceutically acceptable salt thereof, and a pharmaceutically acceptable carrier.

### DETAILED DESCRIPTION OF THE INVENTION

Hereinafter, the present invention is described in detail.

As used herein, the term “alkyl” refers to a straight or branched chain saturated hydrocarbon radical, which is optionally substituted by one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “carbocyclic ring” refers to a monocyclic or fused bicyclic hydrocarbon ring composed of 5 to 9 carbon atoms. Five-to nine-membered rings may contain at least one double bond in the ring structure. Exemplary carbocyclic rings include, but are not limited to, cyclopropane, cyclobutane, cyclopentane, cyclopentenane, cyclohexane, and cycloheptane. Exemplary fused bicyclic rings include, but are not limited to, decahydronaphthalene. A carbocyclic ring can be optionally substituted with one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, aryl, and aryloxy.

As used herein the term “heterocyclic ring” refers to a heteromonocyclic or heterobicyclic ring.

As used herein the term “heteromonocyclic ring” refers to a monocyclic ring which has atoms of at least one heteroatom such as nitrogen, oxygen and sulfur as members of its ring. A heteromonocyclic ring is saturated or unsaturated, and the unsaturated ring may have aromaticity. Exemplary heteromonocyclic rings include, but are not limited to, pyrrolidine, oxolane, thiolane, pyrrole, furan, thiophene, piperidine, oxane, thiane, pyridine, pyran, thiopyran, azepane, oxepane, thiepane, azepine, oxepine, thiepine, diazepine, thiazepine, azocane, oxecane, thiocane, azocine, oxazine, oxazepine, dioxine, and pyrazine.

As used herein the term “heterobicyclic ring” refers to a fused bicyclic ring which has atoms of at least one heteroatom such as nitrogen, oxygen and sulfur as members of its rings. A heterobicyclic ring is saturated or unsaturated, and the unsaturated ring may have aromaticity. Preferably, a heteromonocyclic ring comprises 5 to 5 carbon atoms and 1 to 3 heteroatoms, referred to herein as “C₂₋₅ heteroaryl”. Exemplary heterobicyclic rings include, but are not limited to, quinoxaline, phenazine, carbazole, indole, isoindole, quinoline, isoquinoline, benzazepine, and acridine.

A heteromonocyclic or heterobicyclic ring is optionally substituted by at least one substituent such as halogen, hydroxy, alkyl, alkoxy, aryl, and heteroaryl.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic group, aryl, alkoxy optionally having one to three fluorine substituents, aryloxy, aralkoxy, acyl, aroyl, heteroaroyl, acyloxy, and aroyloxy. Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl and phenanthryl, as well as substituted derivatives thereof.

As used herein, the term “heteroaryl” refers to a monocyclic- or polycyclic aromatic ring comprising carbon atoms, hydrogen atoms, and one or more heteroatoms, preferably, 1 to 3 heteroatoms, independently selected from nitrogen, oxygen, and sulfur. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, phenyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “C₂₋₅ heteroaryl”.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein the term “aralkoxy” refers to the group —ORₐR_{b}, wherein Rₐ is alkyl and R_{b} is aryl as defined above.

As used herein the term “aryloxy” refers to the group —OR_{b}, wherein R_{b} is aryl as defined above.

It is to be understood that the present invention also includes a pharmaceutically acceptable salt and an acid addition salt of the inventive compound, such as a hydrochloride, trifluoroacetic acid, formic acid, citric acid, fumaric acid, fumarate mono-sodium, p-toluenesulfonic acid, stearic acid, citrate di-sodium, tartaric acid, malic acid, lactic acid, succinic acid, or salicylic acid addition salt.

Further, the present invention also includes a prodrug form of the inventive compound. A prodrug is an active or inactive compound that is modified chemically through in vivo physiological action, such as hydrolysis, metabolism and the like, into a compound of the present invention following administration of the prodrug to a patient

The compounds of the present invention may contain one or more asymmetric carbon atoms and may exist in racemic and optically active forms. All of these compounds and diastereomers are incorporated within the scope of the present invention.

In one embodiment of the present invention, the chemical structure of the inventive compounds is represented by the following formula (Ia).

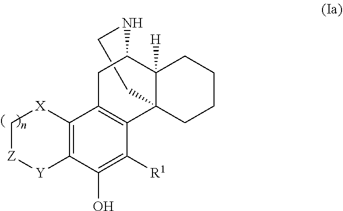

wherein, n is 1 or 2; R¹ is H, halogen, C₁₋₃ alkyl, or C₁₋₂ alkoxy; X and Y are each independently —NH—, —N(R²)—, —N(C(═O)R²)— or —O—; Z is —CH₂— or —CH(R²)—; Y and Z are optionally connected together to form 5 to 9-membered carbocyclic or heterocyclic ring; and R² is H, C₁₋₄ alkyl, C₆₋₁₂ aryl, or C₃₋₁₁ heteroaryl.

In a further preferable embodiment, the chemical structure of such compounds is represented by the following formula (Ia′).

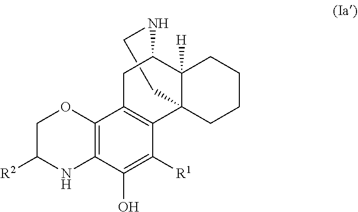

wherein, R¹ is H, halogen, C₁₋₃ alkyl, or C₁₋₂ alkoxy; and R² is H, C₁₋₄ alkyl, C₆₋₁₂ aryl, or C₃₋₁₁ heteroaryl.

In another embodiment of the present invention, the chemical structure of such compounds is represented by the following formula (Ib).

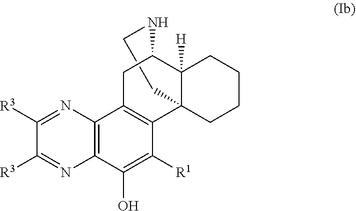

wherein, R¹ is H, halogen, C₁₋₃ alkyl, or C₁₋₂ alkoxy; and R³ is H, C₁₋₄ alkyl, C₆₋₁₂ aryl, or C₃₋₁₁ heteroaryl.

In a further preferable embodiment, the chemical structure of such compounds is represented by the following formula (Ib′).

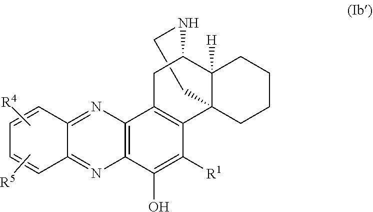

wherein, R¹ is H, halogen, C₁₋₃ alkyl, or C₁₋₂ alkoxy; and R⁴ and R⁵ are each independently H, C₁₋₄ alkyl, C₆₋₁₂ aryl, or C₃₋₁₁ heteroaryl.

In another embodiment of the present invention, the chemical structure of such compounds is represented by the following formula (Ic).

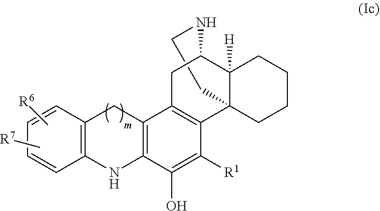

wherein, m is an integer ranging from 0 to 3; R¹ is H, halogen, C₁₋₃ alkyl, or C₁₋₂ alkoxy; and R⁶ and R⁷ are each independently H, halogen, hydroxy, C₁₋₄ alkyl, C₁₋₄ alkoxy, C₆₋₁₂ aryl, or C₃₋₁₁ heteroaryl.

Exemplary compounds in the present invention are selected from the group consisting of:
(6S,6aS,10aS)-2,3,4,5,6,6a,7,8,9,10-Decahydro-6,10a-(epiminoethano)phenanthro[2,1-b][1,4]oxazin-12-ol trifluoroacetic acid (TFA) salt;
(7S,7aS,11aS)-3,4,5,6,7,7a,8,9,10,11-Decahydro-2H-7,11a-(epiminoethano)phenanthro[2,1-b][1,4]oxazepin-13-ol TFA salt;
1-((6S,6aS,10aS)-12-Hydroxy-2,3,6,6a,7,8,9,10-octahydro-6,10a-(epiminoethano)phenanthro[2,1-b][1,4]oxazin-4(5H)-yl)ethanone TFA salt;
(6S,6aS,10aS)-4-Methyl-2,3,4,5,6,6a,7,8,9,10-decahydro-6,10a-(epiminoethano)phenanthro[2,1-b][1,4]oxazin-12-ol TFA salt;
(6S,6aS,10aS)-3,5,6,6a,7,8,9,10-Octahydro-2H-6,10a-(epiminoethano)phenanthro[1,2-b][1,4]dioxin-12-ol TFA salt;
(6S,6aS,10aS)-2-Methyl-1,2,3,5,6,6a,7,8,9,10-decahydro-6,10a-(epiminoethano)phenanthro[1,2-b][1,4]oxazin-12-ol TFA salt;
(6S,6aS,10aS)-1,2,3,5,6,6a,7,8,9,10-Decahydro-6,10a-(epiminoethano)phenanthro[1,2-b][1,4]oxazin-12-ol TFA salt;
(6S,6aS,10aS)-2-Phenyl-1,2,3,5,6,6a,7,8,9,10-decahydro-6,10a-(epiminoethano)phenanthro[1,2-b][1,4]oxazin-12-ol TFA salt;
(6S,6aS,10aS)-6,6a,7,8,9,10-Hexahydro-5H-6,10a-(epiminoethano)naphtho[2,1-f]quinoxalin-12-ol TFA salt;
(4aS,14S,14aS)-2,3,4,13,14,14a-Hexahydro-1H-14,4a-(epiminoethano)naphtho[2,1-a]phenazin-6-ol TFA salt;
(4aS,13S,13aS)-1,2,3,4,7,12,13,13a-Octahydro-13,4a-(epiminoethano)naphtho[1,2-c]carbazole-6,8-diol TFA salt;
(4aS,13S,13aS)-1,2,3,4,7,12,13,13a-Octahydro-13,4a-(epiminoethano)naphtho[1,2-c]carbazol-6-ol TFA salt;
(4aS,13S,13aS)-1,2,3,4,7,12,13,13a-Octahydro-13,4a-(epiminoethano)naphtho[1,2-c]carbazole-6,10-diol TFA salt; and
(4aS,13S,13aS)-10-Methyl-1,2,3,4,7,12,13,13a-octahydro-13,4a-(epiminoethano)naphtho[1,2-c]carbazol-6-ol TFA salt.

### General Synthesis of the Compounds of Formula (I)

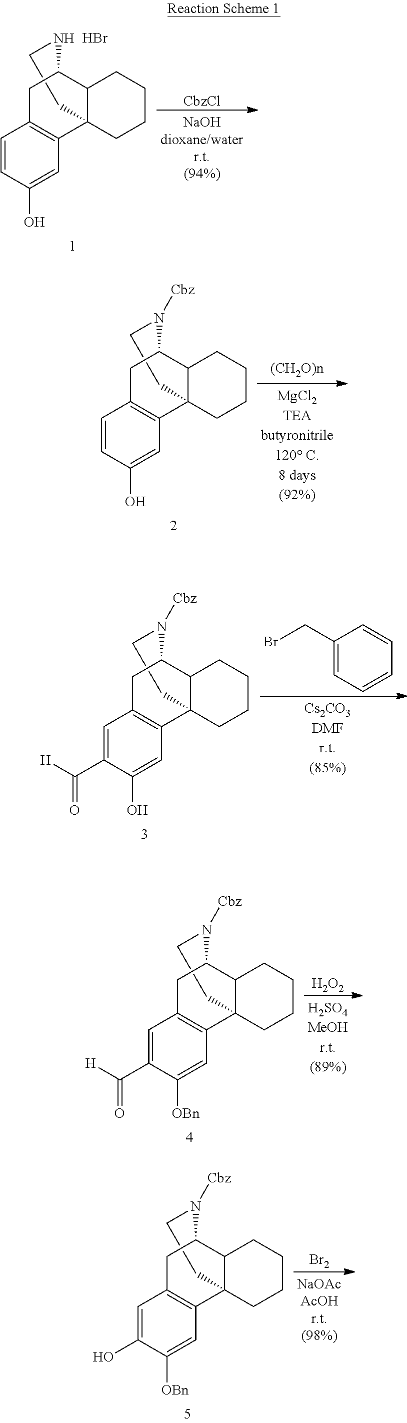

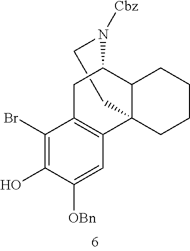 [SMILES: O=C(OCc1ccccc1)N1CC[C@@]23CCCCC2[C@@H]1Cc1c3cc(OCc2ccccc2)c(O)c1Br]

As shown in Reaction Scheme 1, o-formylation is conducted on compound 2 by adopting a known procedure (10 eq of paraformaldehyde, 1.5 eq of MgCl₂ and 2.5 eq of triethylamine in heated butyronitrile for 8 days [Hansen, T. V. et al. Tetrahedron Lett. 2005, 46, 3357-3358]) to aldehyde 3. After benzylation at C-3, formyl group of structure 4 is transformed into the corresponding hydroxy group 5 by use of hydrogen peroxide in the presence of conc. sulfuric acid [Mark, C. et al. J. Med. Chem. 1997, 40, 2323-2334]. o-Bromination is accomplished by adopting a known procedure by using bromine in the presence of sodium acetate in a solvent such as acetic acid to afford compound 6 as a yellow gum in high yield (98%).

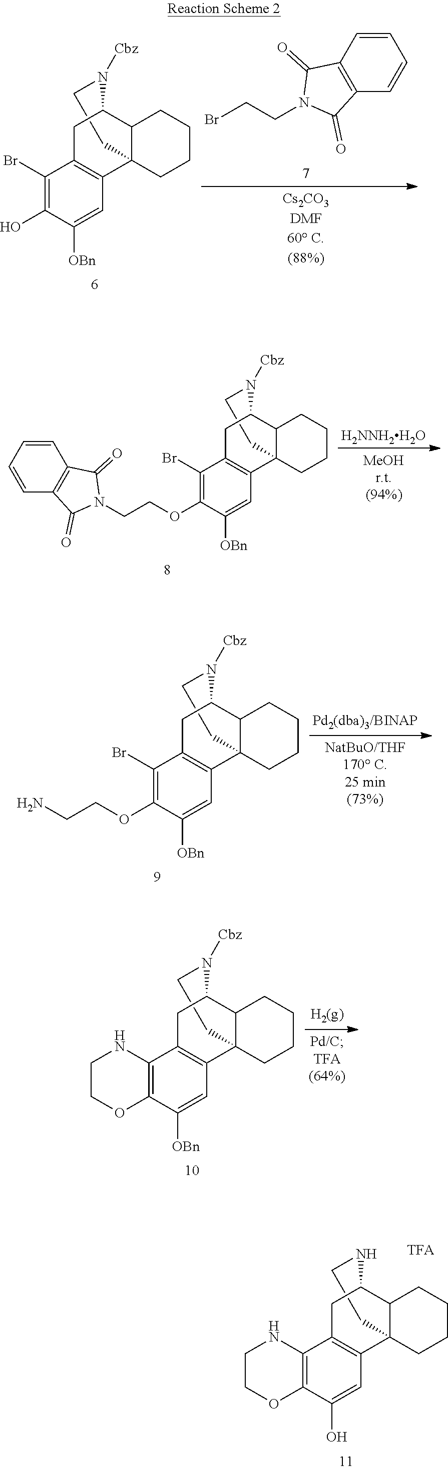

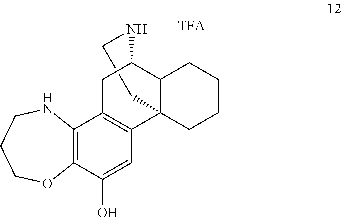 [SMILES: Oc1cc2c(c3c1OCCCN3)C[C@@H]1NCC[C@]23CCCCC13]

As shown in Reaction Scheme 2, 2-(2-bromoethyl)isoindoline-1,3-dione 7 is treated with alcohol 6 in the presence of cesium carbonate in a solvent such as DMF to produce compound 8 as a white solid in 88% yield. Hydrazinolysis of compound 8, followed by cyclization by use of sodium tert-butoxide in the presence of a catalytic amount of Pd₂(dba)₃, 2,2′-bis(diphenylphosphino)-1,1′-binaphthyl (BINAP) in THF [Bower, J. F. et al. Org. Lett. 2007, 9, 3283-3286] give rise to the corresponding pentacycle 10 as a yellow solid in 78% yield. Hydrogenation of compound 10 with Pd on carbon in the presence of a suitable solvent such as isopropyl alcohol, and subsequent purification by reverse-phase prep HPLC (0.1% TFA) generate the inventive target product 11 as a white solid in 64% yield. The corresponding 7-membered ring 12 is obtained by following a similar pathway shown here by using 2-(2-bromopropyl)isoindoline-1,3-dione 13 instead of using 2-(2-bromoethyl)isoindoline-1,3-dione 7.

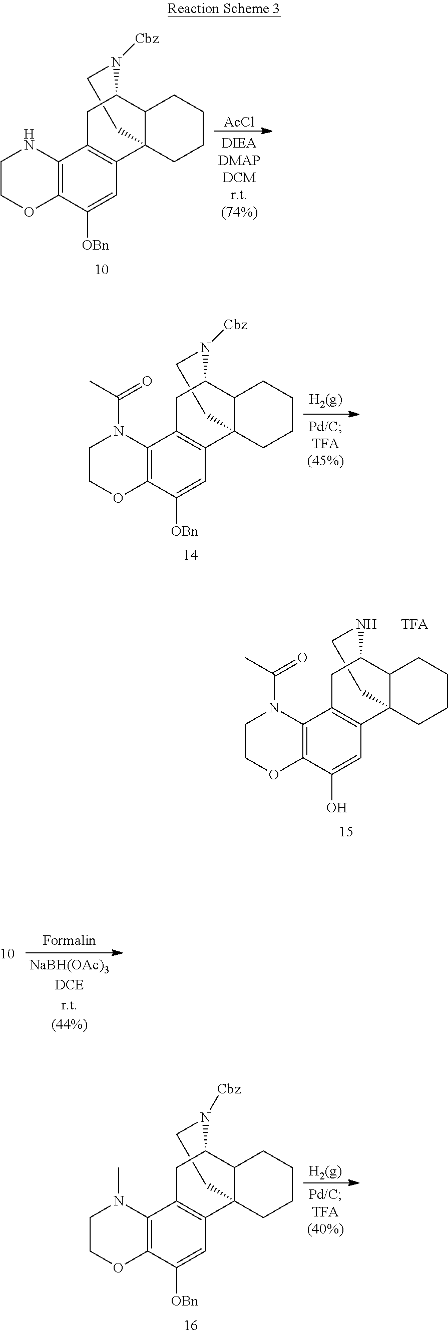

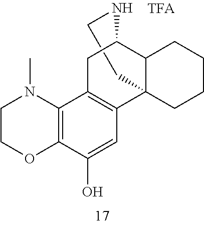 [SMILES: CN1CCOc2c(O)cc3c(c21)C[C@@H]1NCC[C@]32CCCCC12]

As shown in Reaction Scheme 3, the intermediate 10 may be further utilized to generate a variety of derivatives. First, acetylation of compound 10 with acetyl chloride in the presence of N,N-diisopropylethylamine (DIEA), 4-dimethylaminopyridine (DMAP) in DCM produces the corresponding acetylated compound 14 as brown gum. Second, hydrogenation of the resulting compound 14 with Pd on carbon affords the inventive target compound 15 as a yellow gum after purification by prep HPLC (0.1% TFA). Also, compound 10 is treated with formalin in the presence of sodium triacetoxyborohydride to give methylated product 16. Hydrogenation followed by purification by prep HPLC (0.1% TFA) produces another target compound 17 as a yellow gum in 40% yield.

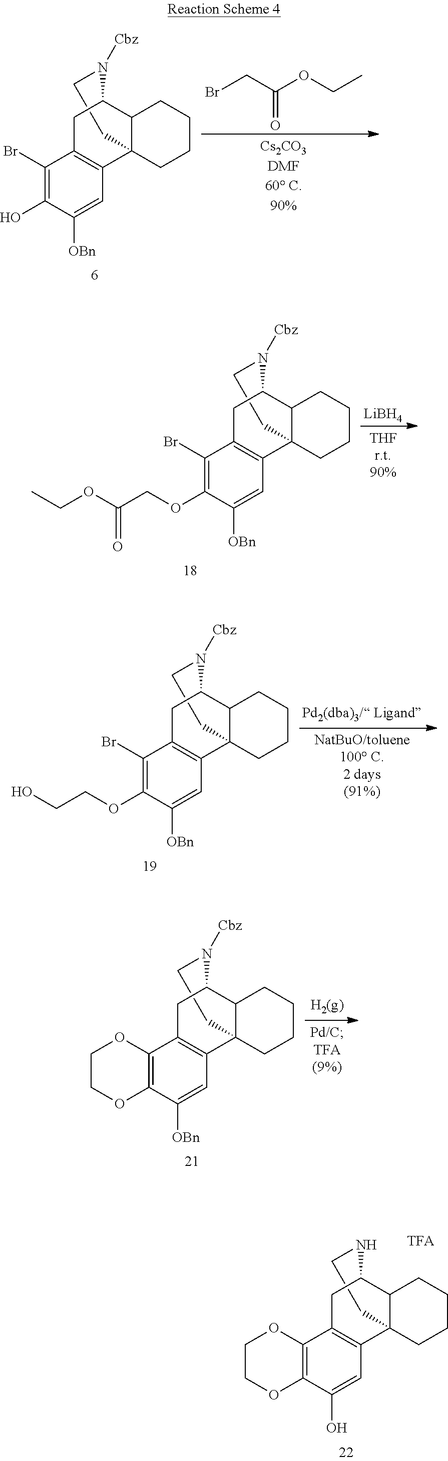

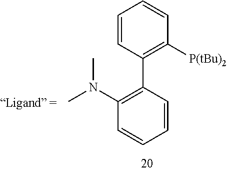

As shown in Reaction Scheme 4, another type of cyclization product may be obtained. Thus, treatment of alcohol 6 with ethyl 2-bromoacetate in the presence of cesium carbonate in a suitable solvent such as DMF produces the corresponding ester 18 as a yellow gum in 90% yield. Reduction of ester 18 with lithium borohydride gives alcohol 19, which is subsequently used for intramolecular cyclization by using sodium tert-butoxide in the presence of a catalytic amount of Pd₂(dba)₃ and a catalytic amount of ligand 20 in heated toluene for two days to provide the pentacycle 21 [Shin-itsu, K. et al. J. Am. Chem. Soc. 2001, 123, 12202-12206]. Hydrogenation followed by purification by prep HPLC (0.1% TFA) produces another target compound 22 as colorless gum in 9% yield.

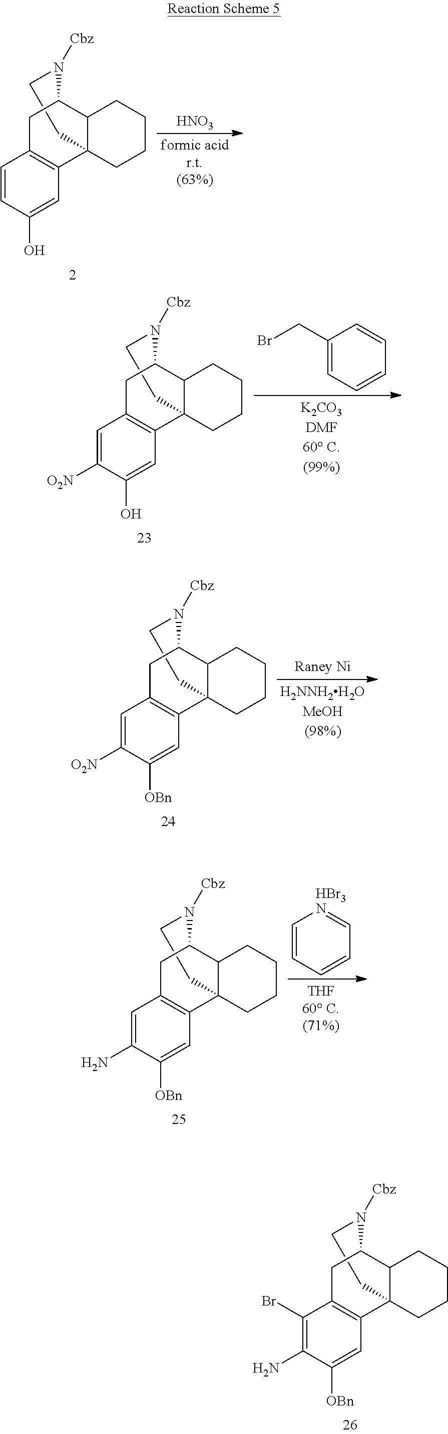

Other type of HM-based polycycle may be also obtained by using a sequence disclosed in Reaction Scheme 5. Thus, treatment of phenol 2 with a mixture of nitric acid and formic acid produces the nitro-phenyl 23 as a yellow solid in 63% yield. Benzylation and subsequent reduction (Raney Ni, hydrazine) provide the corresponding aniline 25 as white solid in almost quantitative yield [Metzler, M. et al. Tetrahedron 1971, 27, 2225-2246]. Treatment of intermediate 25 with pyridinium tribromide in a suitable solvent such as THF produces the corresponding bromide 26 as a yellow solid in 71% yield.

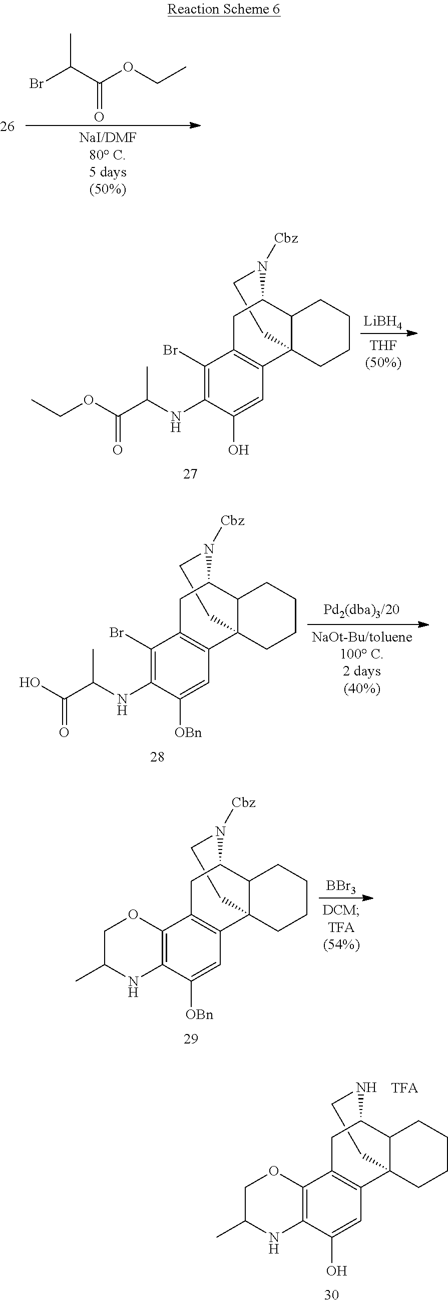

As shown in Reaction Scheme 6, compound 26 is treated with ethyl 2-bromopropanoate and sodium iodide in a suitable solvent such as DMF to produce the corresponding ester 27. Reduction of ester 27 with lithium borohydride gives alcohol 28, which is subsequently used for intramolecular cyclization by use of sodium tert-butoxide in the presence of a catalytic amount of Pd₂(dba)₃, a catalytic amount of ligand 20 in heated toluene for two days to provide the pentacycle 29 [Shin-itsu, K. et al. J. Am. Chem. Soc. 2001, 123, 12202-12206]. Treatment of 29 with boron tribromide followed by purification by prep HPLC (0.1% TFA) produces another target compound 30 as off-white solid in 54% yield.

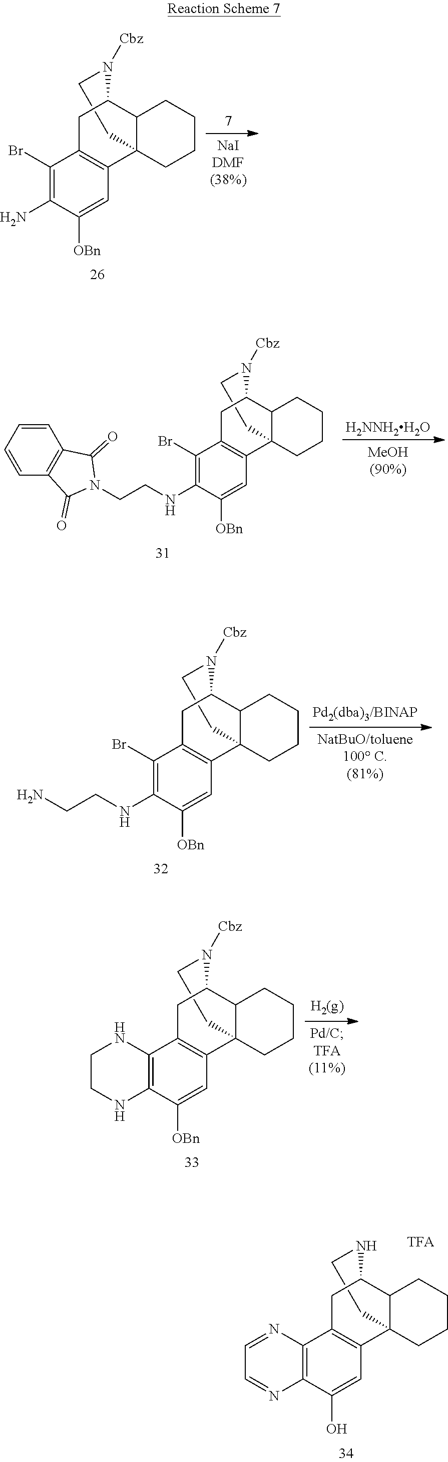

As shown in Reaction Scheme 7, 2-(2-bromoethyl)isoindoline-1,3-dione 7 is treated with aniline 26 in the presence of sodium iodide in a solvent such as DMF to produce compound 31 as a yellow solid in 38% yield. Hydrazinolysis (90%) of compound 8, followed by cyclization (81%) by use of sodium tert-butoxide in the presence of a catalytic amount of Pd₂(dba)₃, BINAP in THF [Bower, J. F. et al. Org. Lett. 2007, 9, 3283-3286] give rise to the pentacycle 33 as a yellow solid in 45% yield. Hydrogenation of compound 33 with Pd on carbon, and subsequent purification by reverse-phase prep HPLC (0.1% TFA) generate the inventive target product 34 as a yellow solid in 11% yield. This product is formed by way of concurrent oxidation toward aromaticity.

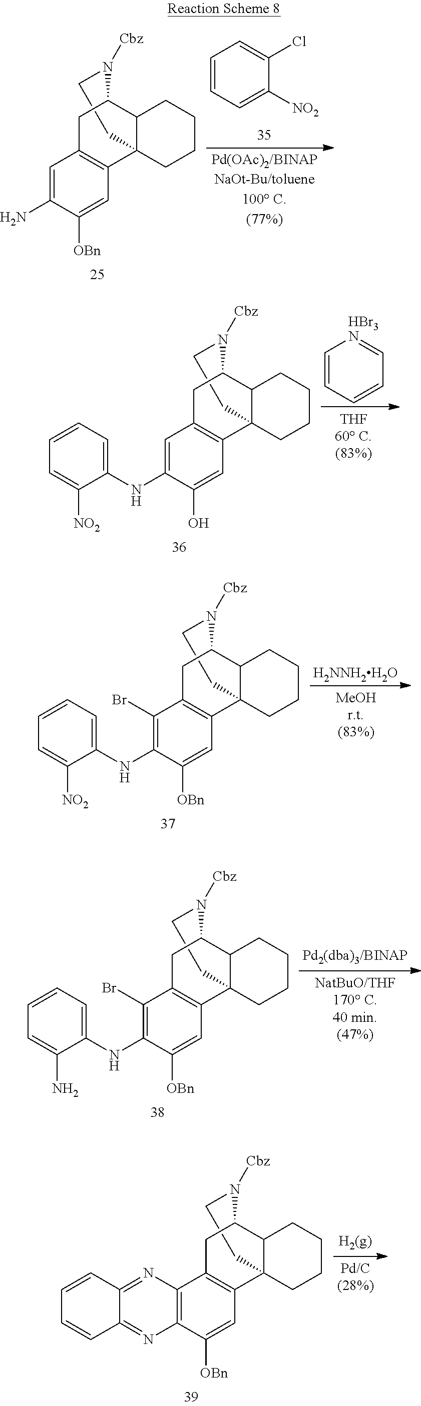

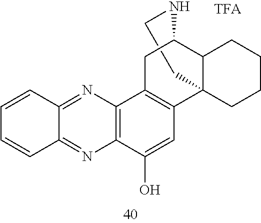 [SMILES: Oc1cc2c(c3nc4ccccc4nc13)C[C@@H]1NCC[C@]23CCCCC13]

Another synthetic variation of this series is shown in Reaction Scheme 8. Thus, aniline 25 is treated with 1-chloro-2-nitrobenzene 35 by use of sodium tert-butoxide in the presence of a catalytic amount of Pd(OAc)₂, BINAP in a suitable solvent such as toluene to provide compound 36 as a yellow solid in 77% yield. Treatment of 36 with pyridinium tribromide followed by reduction of nitro group with Raney Nickel and hydrazine provides the corresponding aniline 38 as a yellow solid in 83% yield. Intramolecular cyclization of compound 37 under the conditions of sodium tert-butoxide in the presence of a catalytic amount of Pd₂(dba)₃, BINAP in THF gives rise to the hexacycle 39 as a yellow solid in 47% yield. This product is formed most likely by way of initial cyclization and subsequent oxidation toward aromaticity. Hydrogenation of compound 39 with Pd on carbon, and subsequent purification by reverse-phase prep HPLC (0.1% TFA) generate the inventive target product 40 as a yellow solid in 28% yield.

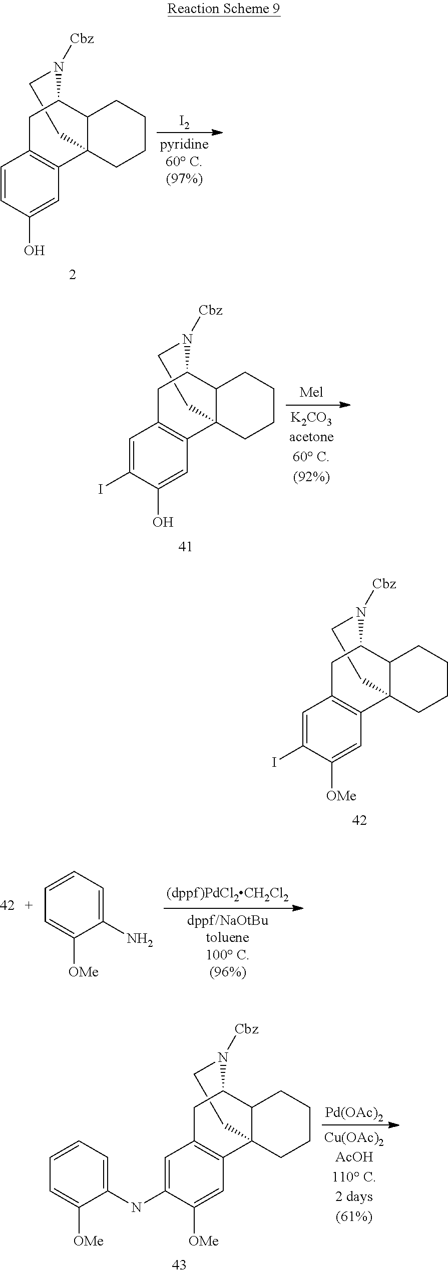

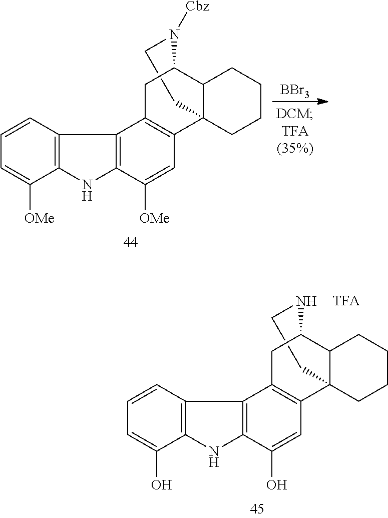

Other type of polycycle ring may be formed as in Reaction Scheme 9. Thus, iodide 41 is produced by treatment of alcohol 2 with iodine in pyridine as a yellow solid in 97% yield. Methylated compound 42 is coupled with 2-methoxyaniline smoothly under the conditions of sodium tert-butoxide in the presence of a catalytic amount of (dppf)PdCl₂, dppf in toluene to produce compound 43 as a yellow solid. Intramolecular oxidative cyclization is conducted on compound 43 under the conditions of palladium(II) acetate and copper(II) acetate in a suitable solvent such as acetic acid to generate hexacycle 44 as a yellow solid in 61% yield [Choi, T. A. et al. Med. Chem. Res. 2008, 17, 374-385]. Treatment of 44 with boron tribromide (simultaneous deprotection of Cbz group and bis-demethylation) followed by purification by prep HPLC (0.1% TFA) produces another target compound 45 as brown solid in 35% yield.

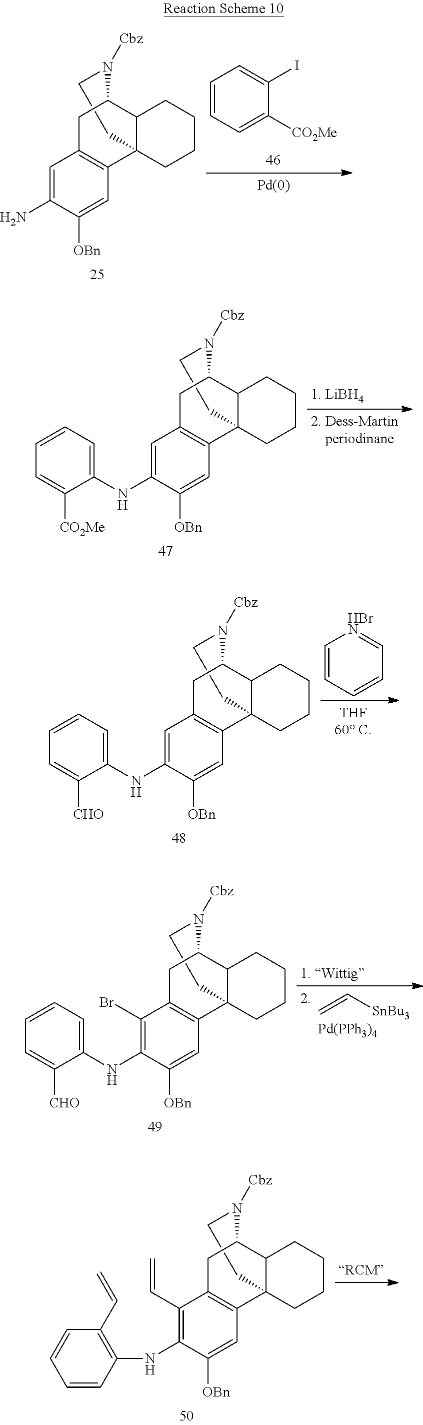

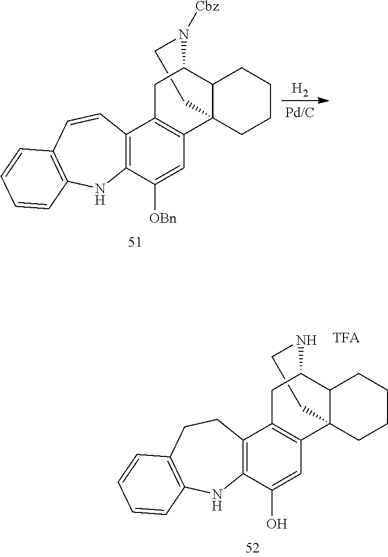

Another type of polycycle derivatives may be formed as in Reaction Scheme 10. Thus, ester 47 is produced by coupling aniline 25 with methyl 2-iodobenzoate 46 in the presence of catalytic Pd(0). Ester 47 is reduced with lithium borohydride and oxidized with Dess-Martin periodinane to generate the corresponding aldehyde 48, which is treated with pyridinium tribromide to produce bromide 49. Two-step conversion of bromide 49 to divinyl compound 50 is accomplished by using initial Wittig reaction and subsequent Stille coupling reaction. Ring-closing olefin metathesis of divinyl compound 50 may provide another type of polycycle compound which would be hydrogenated and purified by reverse-phase prep HPLC to generate an inventive target compound of structure 52.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates may involve nonaqueous solvents such as acetone, ethanol, n-propanol, isopropanol, n-butanol, tert-butanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The present invention also provides a pharmaceutical composition for treating or preventing a neurodegenerative disease, comprising the compound of the present invention and a pharmaceutically acceptable carrier. The neurodegenerative disease may be selected from the group consisting of Alzheimer's disease, Parkinson's disease, Huntington's disease, amyotrophic lateral sclerosis, and ischemic stroke.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carrier routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 to 500 mg/kg body weight, and preferably from 1 to 100 mg/kg body weight of the compound of formula (I) or a prodrug or a pharmaceutically acceptable salt thereof.

The suitable daily dosage for oral administration is about 0.1 to 500 mg/kg body weight of the compound of formula (I) or a prodrug or a pharmaceutically acceptable salt thereof, may be administered 1 to 3 times a day or every two days, depending on the patient's condition.

The present invention further provides a method for treating or preventing a neurodegenerative disease, comprising administering to a patient in need of treatment thereof the compound of formula (I) or a prodrug or a pharmaceutically acceptable salt thereof.

The present invention further provides a use of the compound of formula (I) or a prodrug or a pharmaceutically acceptable salt thereof for the manufacture of a medicament for preventing or treating a neurodegenerative disease.

The present invention is further described and illustrated in Examples provided below, which are, however, not intended to limit the scope of the present invention.

### EXAMPLE

Unless otherwise noted, all starting materials were obtained from commercial suppliers and used without further purification.

As used herein, the symbols and conventions used describing the processes, schemes and examples of the present invention are consistent with those used in the contemporary scientific literature, for example, the Journal of the American Chemical Society or the Journal of Biological Chemistry. The following abbreviations are used in the Examples:
Hz (Hertz);
TLC (thin layer chromatography);
Tᵣ (retention time);
RP (reverse phase);
MeOH (methanol);
i-PrOH (isopropanol);
TFA (trifluoroacetic acid);
TEA (triethylamine);
EtOH (ethanol);
THF (tetrahydrofuran);
DMSO (dimethylsulfoxide);
EtOAc (ethyl acetate);
DCM (dichlromethane);
HOAc (acetic acid);
DMF (N,N-dimethylformamide);
Ac (acetyl);
CDI (1,1-carbnyldiimidazole);
Bn (benzyl);
HOSu (N-hydroxysuccinimide);
HOBT (1-hydroxybenzotriazole);
Boc (tert-butyloxycarbonyl);
mCPBA (meta-chloroperbenzoic acid);
FMOC (9-fluorenylmethoxycarbonyl);
DCC (dicyclohexylcarbodiimide);
Cbz (benzyloxycarbonyl);
NMM (N-methyl morpholine);
HOAt (1-hydroxy-7-azabenzotriazole);
TBAF (tetra-n-butylammonium fluoride);
THP (tetrahydro-2H-pyran-2-yl);
DMAP (4-dimethylaminopyridine);
HPLC (high pressure liquid chromatography);
BOP (bis(2-oxo-3-oxazolidinyl)phosphinic chloride);
EDCI (1-ethyl-3-[3-dimethylaminopropyl]carbodiimide hydrochloride);
HBTU (O-Benzotriazolel-yl-N,N,N′,N′-tetramethyluronium hexafluorophosphate);
DBU (1,8-diazabicyclo[5.4.0]undec-7-ene);
IPA (2-propanol);
NIS (N-iodosuccinimide);
NFPT (1-fluoropyridinium triflate);
DAST (diethylaminosulfur trifluoride);
BINAP (racemic-2,2′-bis(diphenylphosphino)-1,1′-binaphthyl); and
dppf (1,1′-bis(diphenylphosphino)ferrocene).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted, and all solvents are of the highest available purity unless otherwise indicated.

Microwave reaction was conducted with a Biotage microwave reactor.

¹H NMR spectra were recorded on a spectrometer (ECX-400 or JNM-LA300, Jeol Ltd.) Chemical shifts were expressed in parts per million (ppm, 6 units). Coupling constants are in units of hertz (Hz). Splitting patterns describe apparent multiplicities and are designated as s (singlet), d (doublet), t (triplet), q (quartet), quint (quintet), m (multiplet), and br (broad).

Mass spectra were obtained with either a Micromass, Quattro LC Triple Quadrupole Tandem Mass Spectometer, ESI or Agilent, 6110 Quadrupole LC/MS, ESI.

For preparative HPLC, ca 100 mg of a product was injected in 1 mL of DMSO onto a SunFire™ Prep C18 OBD 5 um 19×100 mm Column with a 10 min gradient from 10% CH₃CN to 90% CH₃CN in H₂O. Flash chromatography was carried using Merck silica gel 60 (230-400 mesh). Most of the reactions were monitored by thin-layer chromatography on 0.25 mm E. Merck silica gel plates (60E-254), visualized with UV light using a 5% ethanolic phosphomolybdic acid or p-anisaldehyde solution.

### Example 1

### (6S,6aS,10aS)-2,3,4,5,6,6a,7,8,9,10-Decahydro-6,10a-(epiminoethano)phenanthro[2,1-b][1,4]oxazin-12-ol TFA salt (11)

### Step 1

### (4bS,8aS,9S)-Benzyl 3-hydroxy-6,7,8,8a,9,10-hexahydro-5H-9,4-b-(epiminoethano)phenanthrene-11-carboxylate (2)

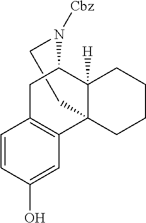 [SMILES: O=C(OCc1ccccc1)N1CC[C@@]23CCCC[C@@H]2[C@@H]1Cc1ccc(O)cc13]

To a solution of (4bS,8aS,9S)-6,7,8,8a,9,10-hexahydro-5H-9,4-b-(epiminoethano)phenanthren-3-ol HBr (1) (50.0 g, 154 mmol) and sodium hydroxide (12.3 g, 308 mmol) in 1,4-dioxane (500 mL) and water (500 mL) was added Cbz-Cl (24.2 mL, 170 mmol) dropwise. The reaction mixture was stirred vigorously at r.t. overnight. After the reaction was completed, water (200 mL) was added. The mixture was extracted with diethyl ether (500 mL×2). The combined organics were dried over MgSO₄, filtered, and evaporated under vacuum. The residue was purified by flash column chromatography (Biotage SP1™) to provide the title compound (54.6 g, 94%) as a white solid.

¹H NMR (300 MHz, CDCl₃) δ 7.36-7.32 (m, 5H), 6.91 (m, 1H), 6.76 (s, 1H), 6.62 (m, 1H), 5.17-5.12 (m, 2H), 4.35 (d, J=29.25 Hz, 1H), 3.92-3.82 (m, 1H), 3.11-3.03 (m, 1H), 2.72-2.56 (m, 2H), 2.31-2.28 (m, 1H), 1.63-1.26 (m, 10H), 1.11-1.00 (m, 1H).

MH+378.

### Step 2

### (4bS,8aS,9S)-Benzyl 2-formyl-3-hydroxy-6,7,8,8a,9,10-hexahydro-5H-9,4-b-(epiminoethano)phenanthrene-11-carboxylate (3)

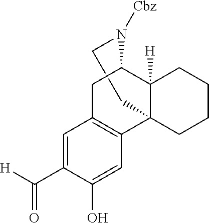 [SMILES: O=Cc1cc2c(cc1O)[C@]13CCCC[C@@H]1[C@H](C2)N(C(=O)OCc1ccccc1)CC3]

A mixture of (4bS,8aS,9S)-benzyl 3-hydroxy-6,7,8,8a,9,10-hexahydro-5H-9,4-b-(epiminoethano)phenanthrene-11-carboxylate (2) (12.0 g, 31.8 mmol), paraformaldehyde (9.55 g, 318 mmol), MgCl₂ (4.54 g, 47.7 mmol) and TEA (11 mL, 79.5 mmol) in butyronitrile (80 mL) was heated at 120° C. for 8 days. The reaction mixture was evaporated to remove the solvent under vacuum. The residue was poured into water (300 mL) and extracted with EtOAc (300 mL×3). The organic phase was dried over MgSO₄ and evaporated under vacuum. The residue was purified by flash column chromatography (Biotage SP1™) to provide the title compound (11.9 g, 92%) as a white solid.

¹H NMR (400 MHz, CDCl₃) δ 10.73 (s, 1H), 9.83 (s, 1H), 7.38-7.26 (m, 5H), 6.95 (s, 1H), 5.18-5.13 (m, 2H), 4.41 (d, J=44.8 Hz, 1H), 3.99-3.87 (m, 1H), 3.13 (td, J=16.0, 5.6 Hz, 1H), 2.78-2.56 (m, 2H), 2.37 (d, J=13.6 Hz, 1H), 1.76-1.54 (m, 5H), 1.49-1.18 (m, 4H), 1.07-0.97 (m, 1H).

MH+406.

### Step 3

### (4bS,8aS,9S)-Benzyl 3-(benzyloxy)-2-formyl-6,7,8,8a,9,10-hexahydro-5H-9,4-b-(epiminoethano)phenanthrene-11-carboxylate (4)

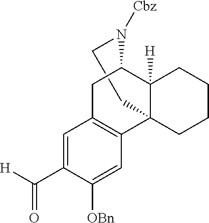 [SMILES: O=Cc1cc2c(cc1OCc1ccccc1)[C@]13CCCC[C@@H]1[C@H](C2)N(C(=O)OCc1ccccc1)CC3]

A mixture of (4bS,8aS,9S)-benzyl 2-formyl-3-hydroxy-6,7,8,8a,9,10-hexahydro-5H-9,4-b-(epiminoethano)phenanthrene-11-carboxylate (3) (10.0 g, 24.7 mmol), benzyl bromide (3.5 mL, 29.6 mmol) and Cs₂CO₃ (12.1 g, 29.6 mmol) in DMF (100 mL) was stirred at r.t. overnight. The reaction mixture was evaporated to remove the solvent under vacuum. The residue was poured into water (300 mL) and extracted with EtOAc (200 mL×2). The organic phase was dried over MgSO₄ and evaporated under vacuum. The residue was purified by flash column chromatography (Biotage SP1™) to provide the title compound (10.4 g, 85%) as a yellow gum.

¹H NMR (400 MHz, CDCl₃) δ 10.50 (s, 1H), 7.57 (d, J=7.6 Hz, 1H), 7.45-7.28 (m, 10H), 6.92 (s, 1H), 5.22-5.12 (m, 4H), 4.38 (d, J=45.2 Hz, 1H), 3.97-3.84 (m, 1H), 3.09 (td, J=18.0, 5.6 Hz, 1H), 2.75-2.56 (m, 2H), 2.26 (d, J=13.6 Hz, 1H), 1.73-1.57 (m, 4H), 1.52-1.46 (m, 2H), 1.38-1.24 (m, 4H), 1.10-0.96 (m, 2H).

MH+409.

### Step 4

### (4bS,8aS,9S)-Benzyl 3-(benzyloxy)-2-hydroxy-6,7,8,8a,9,10-hexahydro-5H-9,4-b-(epiminoethano)phenanthrene-11-carboxylate (5)

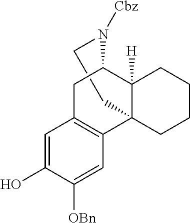 [SMILES: O=C(OCc1ccccc1)N1CC[C@@]23CCCC[C@@H]2[C@@H]1Cc1cc(O)c(OCc2ccccc2)cc13]

To a solution of (4bS,8aS,9S)-benzyl 3-(benzyloxy)-2-formyl-6,7,8,8a,9,10-hexahydro-5H-9,4-b-(epiminoethano)phenanthrene-11-carboxylate (4) (1.64 g, 3.31 mmol) and 30% H₂O₂ (1.5 mL, 14.7 mmol) in MeOH (100 mL) was added conc. H₂SO₄ (0.7 mL, 13.1 mmol) dropwise. The reaction mixture was stirred at r.t. overnight and evaporated under vacuum. The residue was poured into water (300 mL) and extracted with EtOAc (100 mL×3). The organic phase was washed with sat. NaHCO₃ solution (80 mL×3), dried over MgSO₄ and evaporated under vacuum. The residue was purified by flash column chromatography (Biotage SP1™) to provide the title compound (1.43 g, 89%) as a yellow gum.

MH+484.

### Step 5

### (4bS,8aS,9S)-Benzyl 3-(benzyloxy)-1-bromo-2-hydroxy-6,7,8,8a,9,10-hexahydro-5H-9,4-b-(epiminoethano)phenanthrene-1′-carboxylate (6)

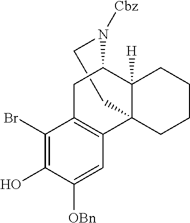 [SMILES: O=C(OCc1ccccc1)N1CC[C@@]23CCCC[C@@H]2[C@@H]1Cc1c3cc(OCc2ccccc2)c(O)c1Br]

To a cooled solution of (4bS,8aS,9S)-benzyl 3-(benzyloxy)-2-hydroxy-6,7,8,8a,9,10-hexahydro-5H-9,4-b-(epiminoethano)phenanthrene-11-carboxylate (5) (400 mg, 3.31 mmol) and NaOAc (135 mg, 1.65 mmol) in glacial AcOH (15 mL) was added Br₂ (85 μL, 1.65 mmol) dropwise. The reaction mixture was stirred at r.t. overnight and evaporated under vacuum. The residue was poured into water (50 mL) and extracted with EtOAc (30 mL×2). The organic phase was washed with sat. Na₂S₂O₃ solution (20 mL×2), dried over MgSO₄ and evaporated under vacuum. The residue was purified by flash column chromatography (Biotage SP1™) to provide the title compound (454 mg, 98%) as a yellow gum.

¹H NMR (400 MHz, CDCl₃) δ 7.43-7.25 (m, 10H), 6.77 (s, 1H), 5.17-5.07 (m, 4H), 4.42 (d, J=46.8 Hz, 1H), 3.89 (ddd, J=33.6, 13.2, 4.0 Hz, 1H), 2.90 (td, J=18.4, 6.0 Hz, 1H), 2.73-2.56 (m, 2H), 2.16 (d, J=13.6 Hz, 1H), 1.67-1.49 (m, 3H), 1.46-1.39 (m, 2H), 1.33-1.23 (m, 3H), 1.08-0.95 (m, 2H).

MH+562.

### Step 6

### (4bS,8aS,9S)-Benzyl 3-(benzyloxy)-1-bromo-2-(2-(1,3-dioxoisoindolin-2-yl)ethoxy)-6,7,8,8a,9,10-hexahydro-5H-9,4-b-(epiminoethano)phenanthrene-11-carboxylate (8)

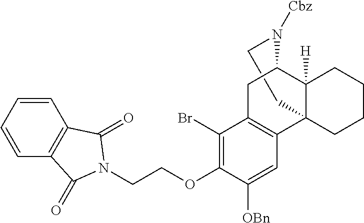 [SMILES: O=C1c2ccccc2C(=O)N1CCOc1c(OCc2ccccc2)cc2c(c1Br)C[C@H]1[C@H]3CCCC[C@@]23CCN1C(=O)OCc1ccccc1]

A mixture of (4bS,8aS,9S)-benzyl 3-(benzyloxy)-1-bromo-2-hydroxy-6,7,8,8a,9,10-hexahydro-5H-9,4-b-(epiminoethano)phenanthrene-11-carboxylate (6) (800 mg, 1.42 mmol), N-(2-bromoethyl)phthalimide (7) (903 mg, 3.56 mmol) and Cs₂CO₃ (579 mg, 1.78 mmol) in DMF (10 mL) was heated at 60° C. overnight. The reaction mixture was evaporated to remove the solvent under vacuum. The residue was poured into water (50 mL) and extracted with EtOAc (30 mL×2). The organic phase was dried over MgSO₄ and evaporated under vacuum. The residue was purified by flash column chromatography (Biotage SP1™) to provide the title compound (722 mg, 88%) as a white solid.

MH+735.

### Step 7

### (4bS,8aS,9S)-Benzyl 2-(2-aminoethoxy)-3-(benzyloxy)-1-bromo-6,7,8,8a,9,10-hexahydro-5H-9,4-b-(epiminoethano)phenanthrene-11-carboxylate (9)

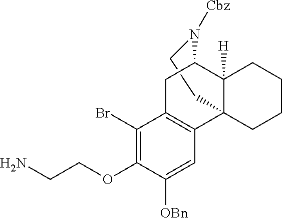 [SMILES: NCCOc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 3-(benzyloxy)-1-bromo-2-(2-(1,3-dioxoisoindolin-2-yl)ethoxy)-6,7,8,8a,9,10-hexahydro-5H-9,4-b-(epiminoethano)phenanthrene-11-carboxylate (8) (722 mg, 0.981 mmol) in MeOH (20 mL) was added hydrazine hydrate (0.14 mL, 2.94 mmol). The reaction mixture was stirred at r.t. overnight and evaporated under vacuum. The residue was purified by flash column chromatography (Biotage SP1™) to provide the title compound (559 mg, 94%) as a white solid.

MH+605.

### Step 8

### (6S,6aS,10aS)-Benzyl 12-(benzyloxy)-2,3,4,5,6,6a,7,8,9,10-decahydro-6,10a-(epiminoethano)phenanthro[2,1-b][1,4]oxazine-15-carboxylate (10)

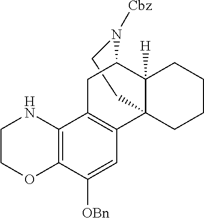 [SMILES: O=C(OCc1ccccc1)N1CC[C@@]23CCCC[C@@H]2[C@@H]1Cc1c3cc(OCc2ccccc2)c2c1NCCO2]

(4bS,8aS,9S)-Benzyl 2-(2-aminoethoxy)-3-(benzyloxy)-1-bromo-6,7,8,8a,9,10-hexahydro-5H-9,4-b-(epiminoethano)phenanthrene-11-carboxylate (9) (81.1 mg, 0.134 mmol) was added to a microwave reactor containing a mixture of Pd₂(dba)₃ (12.3 mg, 0.0134 mmol), BINAP (12.5 mg, 0.0201 mmol) and Sodium t-butoxide (36.0 mg, 0.375 mmol) in THF (10 mL). The capped reactor was placed in a microwave reactor and the mixture was irradiated at 170° C. for 25 min. The reaction mixture was evaporated to remove the solvent under vacuum. The residue was poured into water (20 mL) and extracted with EtOAc (20 mL×2). The organic phase was dried over MgSO₄ and evaporated under vacuum. The residue was purified by flash column chromatography (Biotage SP1™) to provide the title compound (52 mg, 73%) as a yellow gum.

¹H NMR (400 MHz, CDCl₃) δ 7.47-7.23 (m, 10H), 6.26 (s, 1H), 5.18-5.06 (m, 4H), 4.41 (d, J=42.8 Hz, 1H), 4.30 (br s, 2H), 3.90-3.79 (m, 1H), 3.46 (br s, 2H), 2.72-2.54 (m, 2H), 2.23 (t, J=17.2 Hz, 1H), 2.11 (d, J=13.6 Hz, 1H), 1.64-1.15 (m, 8H), 1.09-1.01 (m, 2H).

MH+525.

### Step 9

### (6S,6aS,10aS)-2,3,4,5,6,6a,7,8,9,10-Decahydro-6,10a-(epiminoethano)phenanthro[2,1-b][1,4]oxazin-12-ol TFA salt (11)

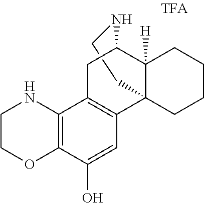 [SMILES: Oc1cc2c(c3c1OCCN3)C[C@@H]1NCC[C@]23CCCC[C@H]13]

To a solution of (6S,6aS,10aS)-benzyl 12-(benzyloxy)-2,3,4,5,6,6a,7,8,9,10-decahydro-6,10a-(epiminoethano)phenanthro[2,1-b][1,4]oxazine-15-carboxylate (10) (120 mg, 0.229 mmol) in IPA (10 mL) was added 10% Pd on charcoal (18 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (77 mg, 81%) as a white solid.

¹H NMR (400 MHz, CD₃OD) δ 6.27 (s, 1H), 4.19 (t, J=4.4 Hz, 2H), 3.72-3.69 (m, 1H), 3.47-3.44 (m, 1H), 3.30-3.28 (m, 2H), 3.05 (dd, J=12.8, 3.2 Hz, 1H), 2.85 (dd, J=18.8, 6.4 Hz, 1H), 2.77 (td, J=13.2, 3.6 Hz, 1H), 2.59 (d, J=18.8 Hz, 1H), 2.33-2.31 (m, 1H), 1.85 (dt, J=12.8, 3.2 Hz, 1H), 1.77-1.65 (m, 2H), 1.56-1.27 (m, 6H), 1.16-1.06 (m, 1H).

MH+301.

### Example 2

### (7S,7aS,11aS)-3,4,5,6,7,7a,8,9,10,11-Decahydro-2H-7,11a-(epiminoethano)phenanthro[2,1-b][1,4]oxazepin-13-ol TFA salt (12)

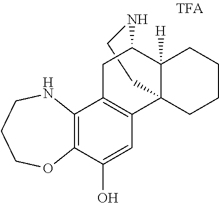 [SMILES: Oc1cc2c(c3c1OCCCN3)C[C@@H]1NCC[C@]23CCCC[C@H]13]

The compound of Example 2 was obtained by repeating the procedure of Example 1 using N-(3-bromopropyl)phthalimide (13) instead of N-(2-bromoethyl)phthalimide (7).

¹H NMR (400 MHz, CD₃OD) δ 4.14-4.13 (m, 1H), 4.07-4.06 (m, 1H), 3.74-3.72 (m, 1H), 3.35-3.33 (m, 2H), 3.06 (dd, J=12.8, 2.8 Hz, 1H), 2.97-2.90 (m, 1H), 2.75 (td, J=13.2, 3.6 Hz, 1H), 2.68-2.63 (m, 1H), 2.36-2.33 (m, 1H), 2.10-1.99 (m, 3H), 1.85 (dt, J=12.4, 2.8 Hz, 1H), 1.78-1.65 (m, 2H), 1.58-1.45 (m, 4H), 1.42-1.21 (m, 4H), 1.16-1.06 (m, 1H).

MH+315.

### Example 3

### 1-((6S,6aS,10aS)-12-Hydroxy-2,3,6,6a,7,8,9,10-octahydro-6,10a-(epiminoethano)phenanthro[2,1-b][1,4]oxazin-4(5H)-yl)ethanone TFA salt (15)

### Step 1

### (6S,6aS,10aS)-Benzyl 4-acetyl-12-(benzyloxy)-2,3,4,5,6,6a,7,8,9,10-decahydro-6,10a-(epiminoethano)phenanthro[2,1-b][1,4]oxazine-15-carboxylate (14)

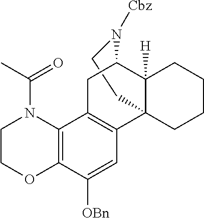 [SMILES: CC(=O)N1CCOc2c(OCc3ccccc3)cc3c(c21)C[C@H]1[C@H]2CCCC[C@@]32CCN1C(=O)OCc1ccccc1]

To a solution of (6S,6aS,10aS)-benzyl 12-(benzyloxy)-2,3,4,5,6,6a,7,8,9,10-decahydro-6,10a-(epiminoethano)phenanthro[2,1-b][1,4]oxazine-15-carboxylate (10) (120 mg, 0.229 mmol), DMAP (33.6 mg, 0.275 mmol) and DIPEA (0.36 mL, 2.06 mmol) in DCM (20 mL) was added acetyl chloride (0.1 mL, 1.37 mmol) at 0° C. The reaction mixture was stirred at 50° C. overnight. The reaction mixture was evaporated to remove the solvent under vacuum. The residue was purified by flash column chromatography (Biotage SP1™) to provide the title compound (96 mg, 74%) as a brown gum.

MH+567.

### Step 2

### 1-((6S,6aS,10aS)-12-Hydroxy-2,3,6,6a,7,8,9,10-octahydro-6,10a-(epiminoethano)phenanthro[2,1-b][1,4]oxazin-4(511)-yl)ethanone TFA salt (15)

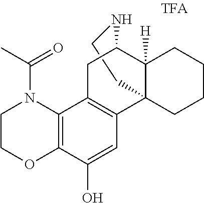 [SMILES: CC(=O)N1CCOc2c(O)cc3c(c21)C[C@@H]1NCC[C@]32CCCC[C@H]12]

To a solution of (6S,6aS,10aS)-benzyl 4-acetyl-12-(benzyloxy)-2,3,4,5,6,6a,7,8,9,10-decahydro-6,10a-(epiminoethano)phenanthro[2,1-b][1,4]oxazine-15-carboxylate (14) (96 mg, 0.169 mmol) in IPA (10 mL) was added 10% Pd on charcoal (10 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (35 mg, 45%) as a yellow gum.

¹H NMR (400 MHz, CD₃OD) δ 6.75 (s, 1H), 4.59-4.55 (m, 1H), 4.37-4.26 (m, 2H), 3.64-3.56 (m, 1H), 3.51-3.45 (m, 1H), 3.19 (dd, J=19.2, 6.4 Hz, 1H), 3.11-3.08 (m, 1H), 3.04-3.00 (m, 1H), 2.58 (td, J=10.4, 3.6 Hz, 1H), 2.47 (d, J=19.6 Hz, 1H), 2.36 (d, J=12.8 Hz, 1H), 2.29-2.28 (m, 3H), 1.88-1.83 (m, 1H), 1.77-1.69 (m, 2H), 1.55-1.27 (m, 7H). MH+343.

### Example 4

### (6S,6aS,10aS)-4-Methyl-2,3,4,5,6,6a,7,8,9,10-decahydro-6,10a-(epiminoethano)phenanthro[2,1-b][1,4]oxazin-12-ol TFA salt (17)

### Step 1

### (6S,6aS,10aS)-Benzyl 12-(benzyloxy)-4-methyl-2,3,4,5,6,6a,7,8,9,10-decahydro-6,10a-(epiminoethano)phenanthro[2,1-b][1,4]oxazine-15-carboxylate (16)

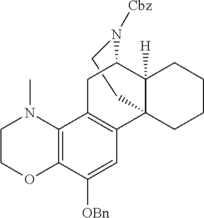 [SMILES: CN1CCOc2c(OCc3ccccc3)cc3c(c21)C[C@H]1[C@H]2CCCC[C@@]32CCN1C(=O)OCc1ccccc1]

To a solution of (6S,6aS,10aS)-benzyl 12-(benzyloxy)-2,3,4,5,6,6a,7,8,9,10-decahydro-6,10a-(epiminoethano)phenanthro[2,1-b][1,4]oxazine-15-carboxylate (10) (148 mg, 0.282 mmol) and formalin (37%, 1.1 mL, 14.1 mmol) in DCE (10 mL) was added sodium triacetoxyborohydride (179 mg, 0.846 mmol). The mixture was stirred at r.t. for 48 hours. The reaction mixture was evaporated to remove the solvent under vacuum. The residue was purified by flash column chromatography (Biotage SP1™) to provide the title compound (67 mg, 44%) as a brown gum.

MH+539.

### Step 2

### (6S,6aS,10aS)-4-Methyl-2,3,4,5,6,6a,7,8,9,10-decahydro-6,10a-(epiminoethano)phenanthro[2,1-b][1,4]oxazin-12-ol TFA salt (17)

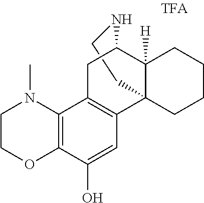 [SMILES: CN1CCOc2c(O)cc3c(c21)C[C@@H]1NCC[C@]32CCCC[C@H]12]

To a solution of (6S,6aS,10aS)-benzyl 12-(benzyloxy)-4-methyl-2,3,4,5,6,6a,7,8,9,10-decahydro-6,10a-(epiminoethano)phenanthr o[2,1-b][1,4]oxazine-15-carboxylate (16) (67 mg, 0.123 mmol) in IPA (10 mL) was added 10% Pd on charcoal (7 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (21 mg, 40%) as a yellow gum.

¹H NMR (400 MHz, CD₃OD) δ 6.59 (s, 1H), 4.29-4.27 (m, 2H), 3.72-3.70 (m, 1H), 3.19-3.09 (m, 3H), 3.06-2.95 (m, 3H), 2.74 (s, 3H), 3.64 (td, J=13.2, 3.6 Hz, 1H), 2.36 (d, J=13.2 Hz, 1H), 1.87 (dt, J=12.8, 3.2 Hz, 1H), 1.77-1.69 (m, 2H), 1.56-1.50 (m, 3H), 1.44-1.26 (m, 3H), 1.20-1.14 (m, 1H).

MH+315.

### Example 5

### (6S,6aS,10aS)-3,5,6,6a,7,8,9,10-Octahydro-2H-6,10a-(epiminoethano)phenanthro[1,2-b][1,4]dioxin-12-ol TFA salt (22)

### Step 1

### (4bS,8aS,9S)-Benzyl 3-(benzyloxy)-1-bromo-2-(2-ethoxy-2-oxoethoxy)-6,7,8,8a,9,10-hexahydro-5H-9,4-b-(epiminoethano)phenanthrene-11-carboxylate (18)

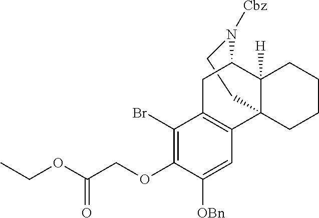 [SMILES: CCOC(=O)COc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 3-(benzyloxy)-1-bromo-2-hydroxy-6,7,8,8a,9,10-hexahydro-5H-9,4-b-(epiminoethano)phenanthrene-1′-carboxylate (6) (500 mg, 0.889 mmol) and Cs₂CO₃ (579 mg, 1.78 mmol) in DMF (10 mL) was added ethyl bromoacetate (0.15 mL, 1.33 mmol). The reaction mixture was stirred at r.t. overnight and evaporated under vacuum. The residue was purified by flash column chromatography (Biotage SP1™) to provide the title compound (520 mg, 90%) as a yellow gum.

MH+648.

### Step 2

### (4bS,8aS,9S)-Benzyl 3-(benzyloxy)-1-bromo-2-(2-hydroxyethoxy)-6,7,8,8a,9,10-hexahydro-5H-9,4-b-(epiminoethano)phenanthrene-11-carboxylate (19)

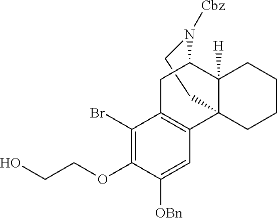 [SMILES: O=C(OCc1ccccc1)N1CC[C@@]23CCCC[C@@H]2[C@@H]1Cc1c3cc(OCc2ccccc2)c(OCCO)c1Br]

To a solution of (4bS,8aS,9S)-benzyl 3-(benzyloxy)-1-bromo-2-(2-ethoxy-2-oxoethoxy)-6,7,8,8a,9,10-hexahydro-5H-9,4-b-(epiminoethano)phenanthrene-11-carboxylate (18) (683 mg, 1.05 mmol) in THF (10 mL) was added lithium borohydride (0.53 mL, 1.05 mmol) at 0° C. The reaction mixture was stirred at r.t. overnight and evaporated under vacuum. The residue was purified by flash column chromatography (Biotage SP1™) to provide the title compound (580 mg, 90%) as a colorless gum.

MH+606.

### Step 3

### (6S,6aS,10aS)-Benzyl 12-(benzyloxy)-3,5,6,6a,7,8,9,10-octahydro-2H-6,10a-(epiminoethano)phenanthro[1,2-b][1,4]dioxine-15-carboxylate (21)

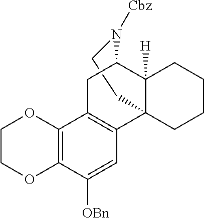 [SMILES: O=C(OCc1ccccc1)N1CC[C@@]23CCCC[C@@H]2[C@@H]1Cc1c3cc(OCc2ccccc2)c2c1OCCO2]

A mixture of (4bS,8aS,9S)-benzyl 3-(benzyloxy)-1-bromo-2-(2-hydroxyethoxy)-6,7,8,8a,9,10-hexahydro-5H-9,4-b-(epiminoethano)phenanthrene-11-carboxylate (19) (300 mg, 0.495 mmol), Pd₂(dba)₃ (4.5 mg, 0.00495 mmol), 2-di-t-butylphosphino-2′-(N,N-dimethylamino)biphenyl (20) (3.4 mg, 0.00990 mmol) and sodium t-butoxide (71.4 mg, 0.743 mmol) in toluene (10 mL) was heated at 100° C. for 2 days and evaporated under vacuum. The residue was purified by flash column chromatography (Biotage SP1™) to provide the title compound (240 mg, 90%) as a brown gum.

MH+526.

### Step 4

### (6S,6aS,10aS)-3,5,6,6a,7,8,9,10-Octahydro-2H-6,10a-(epiminoethano)phenanthro[1,2-b][1,4]dioxin-12-ol TFA salt (22)

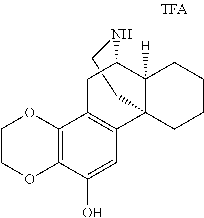 [SMILES: Oc1cc2c(c3c1OCCO3)C[C@@H]1NCC[C@]23CCCC[C@H]13]

To a solution of (6S,6aS,10aS)-benzyl 12-(benzyloxy)-3,5,6,6a,7,8,9,10-octahydro-2H-6,10a-(epiminoethano)phenanthro[1,2-b][1,4]dioxine-15-carboxylate (21) (237 mg, 0.495 mmol) in IPA (10 mL) was added 10% Pd on charcoal (36 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (19 mg, 9%) as a colorless gum.

¹H NMR (400 MHz, CD₃OD) δ 6.41 (s, 1H), 4.30-4.24 (m, 4H), 3.67-3.65 (m, 1H), 3.08-3.04 (m, 1H), 2.96-2.90 (m, 1H), 2.82-2.75 (m, 2H), 2.33 (d, J=12.0 Hz, 1H), 1.86-1.82 (m, 1H), 1.74-1.67 (m, 2H), 1.55-1.27 (m, 7H), 1.12-1.08 (m, 1H).

MH+302.

### Example 6

### (6S,6aS,10aS)-2-Methyl-1,2,3,5,6,6a,7,8,9,10-decahydro-6,10a-(epiminoethano)phenanthro[1,2-b][1,4]oxazin-12-ol TFA salt (30)

### Step 1

### (4bS,8aS,9S)-Benzyl 3-hydroxy-2-nitro-6,7,8,8a,9,10-hexahydro-5H-9,4-b-(epiminoethano)phenanthrene-11-carboxylate (23)

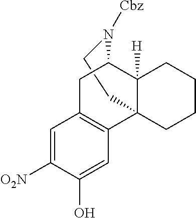 [SMILES: O=C(OCc1ccccc1)N1CC[C@@]23CCCC[C@@H]2[C@@H]1Cc1cc([N+](=O)[O-])c(O)cc13]

To a cooled solution of (4bS,8aS,9S)-benzyl 3-hydroxy-6,7,8,8a,9,10-hexahydro-5H-9,4-b-(epiminoethano)phenanthrene-11-carboxylate (2) (7.46 g, 19.8 mmol) in formic acid (20 mL) was added nitric acid (65%, 1.6 mL, 23.7 mmol) dropwise. The reaction mixture was stirred at r.t. overnight and evaporated to remove the solvent under vacuum. The residue was poured into water (300 mL) and extracted with EtOAc (300 mL×3). The organic phase was washed with sat. NaHCO₃ solution (200 mL×3), dried over MgSO₄ and evaporated under vacuum. The residue was purified by flash column chromatography (Biotage SP1™) to provide the title compound (5.26 g, 63%) as a yellow solid.

¹H NMR (400 MHz, CDCl₃) δ 10.36 (s, 1H), 7.84 (s, 1H), 7.38-7.34 (m, 5H), 7.10 (s, 1H), 5.21-5.13 (m, 2H), 4.42 (d, J=46.8 Hz, 1H), 4.00-3.8 (m, 1H), 3.12 (td, J=16.0, 5.2 Hz, 1H), 2.79-2.54 (m, 2H), 2.36 (d, J=13.6 Hz, 1H), 1.77-1.50 (m, 5H), 1.45-1.17 (m, 4H), 1.02-0.93 (m, 1H).

MH+423.

### Step 2

### (4bS,8aS,9S)-Benzyl 3-(benzyloxy)-2-nitro-6,7,8,8a,9,10-hexahydro-5H-9,4-b-(epiminoethano)phenanthrene-11-carboxylate (24)

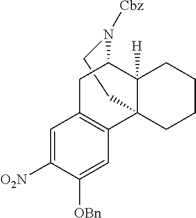 [SMILES: O=C(OCc1ccccc1)N1CC[C@@]23CCCC[C@@H]2[C@@H]1Cc1cc([N+](=O)[O-])c(OCc2ccccc2)cc13]

To a solution of (4bS,8aS,9S)-benzyl 3-hydroxy-2-nitro-6,7,8,8a,9,10-hexahydro-5H-9,4-b-(epiminoethano)phenanthrene-11-carboxylate (23) (11.3 g, 26.7 mmol) and K₂CO₃ (7.38 g, 53.4 mmol) in DMF (100 mL) was added benzyl bromide (3.94 mL, 40.1 mmol). The reaction mixture was heated at 70° C. overnight and evaporated to remove the solvent under vacuum. The residue was poured into water (300 mL) and extracted with EtOAc (150 mL×2). The organic phase was dried over MgSO₄ and evaporated under vacuum. The residue was purified by flash column chromatography (Biotage SP1™) to provide the title compound (13.6 g, 99%) as a white solid.

¹H NMR (400 MHz, CDCl₃) δ 7.62 (s, 1H), 7.45-1.29 (m, 10H), 6.96 (s, 1H), 5.26-5.12 (m, 4H), 4.39 (d, J=46.0 Hz, 1H), 3.97-3.85 (m, 1H), 3.08 (td, J=18.0, 5.6 Hz, 1H), 2.74-2.54 (m, 2H), 2.20 (d, J=14.0 Hz, 1H), 1.73-1.57 (m, 3H), 1.53-1.44 (m, 2H), 1.38-1.24 (m, 3H), 1.02-0.93 (m, 2H). MH+513.

### Step 3

### (4bS,8aS,9S)-Benzyl 2-amino-3-(benzyloxy)-6,7,8,8a,9,10-hexahydro-5H-9,4-b-(epiminoethano)phenanthrene-11-carboxylate (25)

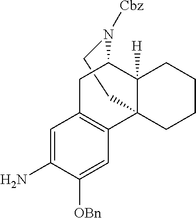 [SMILES: Nc1cc2c(cc1OCc1ccccc1)[C@]13CCCC[C@@H]1[C@H](C2)N(C(=O)OCc1ccccc1)CC3]

To a solution of (4bS,8aS,9S)-benzyl 3-(benzyloxy)-2-nitro-6,7,8,8a,9,10-hexahydro-5H-9,4-b-(epiminoethano)phenanthrene-11-carboxylate (24) (13.6 g, 26.5 mmol) and hydrazine hydrate (12.9 mL, 265 mmol) in MeOH (200 mL) was added Raney Ni (water solution, 1 mL) dropwise. The reaction mixture was stirred at r.t. for 2 hr. and filtered to remove the catalyst. The filtrate was evaporated under vacuum. The residue was further purified by flash column chromatography (Biotage SP1™) to provide the title compound (12.5 g, 98%) as a white solid.

¹H NMR (400 MHz, CDCl₃) δ 7.44-7.30 (m, 10H), 6.69 (s, 1H), 6.45 (d, J=10.8 Hz, 1H), 5.17-5.01 (m, 4H), 4.32 (d, J=40.8 Hz, 1H), 3.96-3.82 (m, 1H), 3.72 (br s, 2H), 3.01 (td, J=17.6, 5.6 Hz, 1H), 2.73-2.52 (m, 2H), 2.20 (d, J=11.6 Hz, 1H), 1.66-1.06 (m, 10H).

MH+483.

### Step 4

### (4bS,8aS,9S)-Benzyl 2-amino-3-(benzyloxy)-1-bromo-6,7,8,8a,9,10-hexahydro-5H-9,4-b-(epiminoethano)phenanthrene-11-carboxylate (26)

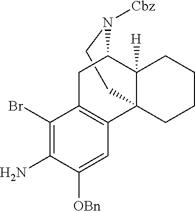 [SMILES: Nc1c(OCc2ccccc2)cc2c(c1Br)C[C@H]1[C@H]3CCCC[C@@]23CCN1C(=O)OCc1ccccc1]

A mixture of (4bS,8aS,9S)-benzyl 2-amino-3-(benzyloxy)-6,7,8,8a,9,10-hexahydro-5H-9,4-b-(epiminoethano)phenanthrene-11-carboxylate (25) (1.29 g, 2.67 mmol) and pyridinium tribromide (1.28 g, 4.01 mmol) in THF (30 mL) was heated at 60° C. overnight and evaporated under vacuum. The residue was further purified by flash column chromatography (Biotage SP1™) to provide the title compound (1.06 g, 71%) as a yellow solid.

¹H NMR (400 MHz, CDCl₃) δ 7.42-7.30 (m, 10H), 6.70 (s, 1H), 5.17-5.01 (m, 4H), 4.41 (d, J=44.8 Hz, 1H), 3.94-3.82 (m, 1H), 2.89 (td, J=16.0, 5.6 Hz, 1H), 2.71-2.64 (m, 2H), 2.17 (d, J=13.6 Hz, 1H), 1.62-1.1.25 (m, 8H), 1.15-0.97 (m, 2H).

MH+561.

### Step 5

### (4bS,8aS,9S)-Benzyl 3-(benzyloxy)-1-bromo-2-((1-ethoxy-1-oxopropan-2-yl)amino)-6,7,8,8a,9,10-hexahydro-5H-9,4-b-(epiminoethano)phenanthrene-11-carboxylate (27)

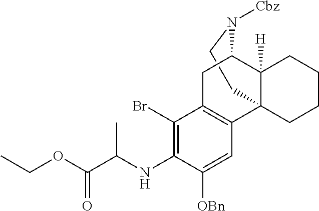 [SMILES: CCOC(=O)C(C)Nc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 2-amino-3-(benzyloxy)-1-bromo-6,7,8,8a,9,10-hexahydro-5H-9,4-b-(epiminoethano)phenanthrene-11-carboxylate (26) (0.98 g, 1.75 mmol) and sodium iodide (0.34 g, 2.27 mmol) in DMF (10 mL) was added ethyl 2-bromopropionate (0.27 mL, 2.09 mmol). The reaction mixture was heated at 80° C. for 5 days and evaporated under vacuum. The residue was further purified by flash column chromatography (Biotage SP1™) to provide the title compound (0.58 g, 50%) as a yellow gum.

MH+661.

### Step 6

### (4bS,8aS,9S)-Benzyl 3-(benzyloxy)-1-bromo-2-((1-hydroxypropan-2-yl)amino)-6,7,8,8a,9,10-hexahydro-5H-9,4b-(epiminoethano)phenanthrene-11-carboxylate (28)

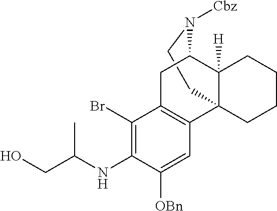 [SMILES: CC(CO)Nc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 3-(benzyloxy)-1-bromo-2-((1-ethoxy-1-oxopropan-2-yl)amino)-6,7,8,8a,9,10-hexahydro-5H-9,4-b-(epiminoethano)phenanthrene-11-carboxylate (27) (0.58 g, 0.877 mmol) in THF (10 mL) was added lithium borohydride (0.88 mL, 1.75 mmol) at 0° C. The reaction mixture was stirred at r.t. overnight and evaporated under vacuum. The residue was purified by flash column chromatography (Biotage SP1™) to provide the title compound (0.27 g, 50%) as a white solid.

MH+619.

### Step 7

### (6S,6aS,10aS)-Benzyl 12-(benzyloxy)-2-methyl-1,2,3,5,6,6a,7,8,9,10-decahydro-6,10a-(epiminoethano)phenanthro[1,2-b][1,4]oxazine-15-carboxylate (29)

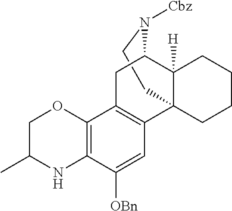 [SMILES: CC1COc2c3c(cc(OCc4ccccc4)c2N1)[C@]12CCCC[C@@H]1[C@H](C3)N(C(=O)OCc1ccccc1)CC2]

A mixture of (4bS,8aS,9S)-benzyl 3-(benzyloxy)-1-bromo-2-((1-hydroxypropan-2-yl)amino)-6,7,8,8a,9,10-hexahydro-5H-9,4b-(epiminoethano)phenanthrene-11-carboxylate (28) (270 mg, 0.436 mmol), Pd₂(dba)₃ (39.9 mg, 0.0436 mmol), 2-di-t-butylphosphino-2′-(N,N-dimethylamino)biphenyl (20) (29.8 mg, 0.0872 mmol) and sodium t-butoxide (62.8 mg, 0.654 mmol) in toluene (5 mL) was heated at 100° C. for 2 days and evaporated under vacuum. The residue was purified by flash column chromatography (Biotage SP1™) to provide the title compound (95 mg, 40%) as a colorless gum.

¹H NMR (400 MHz, CDCl₃) δ 7.44-7.29 (m, 10H), 6.39 (d, J=3.6 Hz, 1H), 5.16-5.01 (m, 4H), 4.37 (d, J=43.2 Hz, 1H), 4.21 (d, J=10.4 Hz, 1H), 3.90-3.76 (m, 2H), 3.47-3.45 (m, 1H), 2.79-2.71 (m, 2H), 2.65-2.61 (m, 1H), 2.20 (d, J=10.8 Hz, 1H), 1.63-1.10 (m, 13H).

MH+539.

### Step 8

### (6S,6aS,10aS)-2-Methyl-1,2,3,5,6,6a,7,8,9,10-decahydro-6,10a-(epiminoethano)phenanthro[1,2-b][1,4]oxazin-12-ol TFA salt (30)

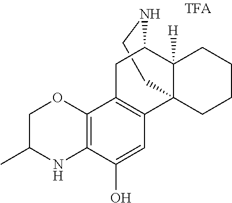 [SMILES: CC1COc2c3c(cc(O)c2N1)[C@]12CCCC[C@@H]1[C@H](C3)NCC2]

To a solution of (6S,6aS,10aS)-benzyl 12-(benzyloxy)-2-methyl-1,2,3,5,6,6a,7,8,9,10-decahydro-6,10a-(epiminoethano)phenanthr o[1,2-b][1,4]oxazine-15-carboxylate (29) (54 mg, 0.101 mmol) in DCM (10 mL) was added BBr₃ (1M in DCM, 0.3 mL, 0.300 mmol) at 0° C. The mixture was stirred at r.t. overnight and quenched by MeOH (1 mL). The reaction mixture was evaporated under vacuum. The residue was further purified by prep. HPLC (Waters, C18 column, 0.1% TFA) to provide the title compound (23 mg, 54%) as a brown solid.

¹H NMR (400 MHz, CD₃OD) δ 6.62 (s, 1H), 4.57-4.52 (m, 1H), 4.12-4.04 (m, 1H), 3.82-3.74 (m, 2H), 3.12 (dd, J=13.2, 4.0 Hz, 1H), 3.00-2.88 (m, 2H), 2.79-2.71 (m, 1H), 2.36 (d, J=14.0 Hz, 1H), 1.97-1.93 (m, 1H), 1.84 (td, J=13.6, 4.8 Hz, 1H), 1.67-1.39 (m, 9H), 1.28-1.18 (m, 1H), 1.05-1.01 (m, 1H).

MH+315.

The following compounds of Example 7 and 8 were obtained by repeating the procedure of Example 6.

### Example 7

### (6S,6aS,10aS)-1,2,3,5,6,6a,7,8,9,10-Decahydro-6,10a-(epiminoethano)phenanthro[1,2-b][1,4]oxazin-12-ol TFA salt (30a)

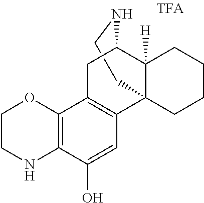 [SMILES: Oc1cc2c(c3c1NCCO3)C[C@@H]1NCC[C@]23CCCC[C@H]13]

¹H NMR (400 MHz, CD₃OD) δ 6.37 (s, 1H), 4.73-4.71 (m, 1H), 4.35 (d, J=12.0 Hz, 1H), 3.90 (m, 1H), 3.63-3.61 (m, 1H), 3.02-3.00 (m, 1H), 2.96-2.90 (m, 1H), 2.85-2.76 (m, 2H), 2.32-2.30 (m, 1H), 1.84-1.81 (m, 1H), 1.73-1.66 (m, 2H), 1.48-1.27 (m, 7H).

MH+301.

### Example 8

### (6S,6aS,10aS)-2-Phenyl-1,2,3,5,6,6a,7,8,9,10-decahydro-6,10a-(epiminoethano)phenanthro[1,2-b][1,4]oxazin-12-ol TFA salt (30b)

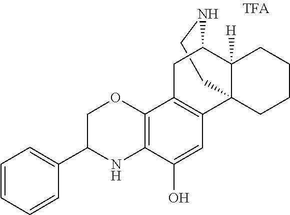 [SMILES: Oc1cc2c(c3c1NC(c1ccccc1)CO3)C[C@@H]1NCC[C@]23CCCC[C@H]13]

¹H NMR (400 MHz, CD₃OD) δ 7.46-7.31 (m, 5H), 6.43 (s, 1H), 4.49-4.45 (m, 1H), 4.37 (dd, J=10.8, 2.8 Hz, 1H), 4.06 (dd, J=10.8, 7.2 Hz, 1H), 3.68-3.66 (m, 1H), 3.08 (dd, J=12.8, 3.2 Hz, 1H), 2.95 (dd, J=19.2, 6.0 Hz, 1H) 2.85-2.79 (m, 2H), 2.35 (d, J=10.4 Hz, 1H), 1.85 (d, J=12.4 Hz, 1H), 1.79-1.70 (m, 2H), 1.57-1.37 (m, 6H), 1.20-1.12 (m, 1H).

MH+377.

### Example 9

### (6S,6aS,10aS)-6,6a,7,8,9,10-Hexahydro-5H-6,10a-(epiminoethano)naphtho[2,1-f]quinoxalin-12-ol TFA salt (34)

### Step 1

### (4bS,8aS,9S)-Benzyl 3-(benzyloxy)-1-bromo-2-((2-(1,3-dioxoisoindolin-2-yl)ethyl)amino)-6,7,8,8a,9,10-hexahydro-5H-9,4-b-(epiminoethano)phenanthrene-11-carboxylate (31)

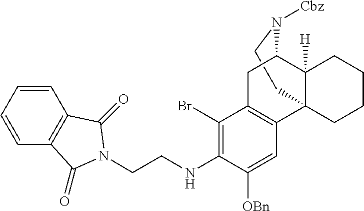 [SMILES: O=C1c2ccccc2C(=O)N1CCNc1c(OCc2ccccc2)cc2c(c1Br)C[C@H]1[C@H]3CCCC[C@@]23CCN1C(=O)OCc1ccccc1]

A mixture of (4bS,8aS,9S)-benzyl 2-amino-3-(benzyloxy)-1-bromo-6,7,8,8a,9,10-hexahydro-5H-9,4-b-(epiminoethano)phenanthrene-11-carboxylate (26) (1.0 g, 1.78 mmol), N-(2-bromoethyl)phthalimide (7) (4.52 g, 17.8 mmol) and sodium iodide (2.67 g, 17.8 mmol) in DMF (20 mL) was heated at 60° C. for 7 days. The reaction mixture was evaporated to remove the solvent under vacuum. The residue was poured into water (100 mL) and extracted with EtOAc (50 mL×2). The organic phase was dried over MgSO₄ and evaporated under vacuum. The residue was purified by flash column chromatography (Biotage SP1™) to provide the title compound (490 mg, 38%) as a yellow solid.

MH+734.

### Step 2

### (4bS,8aS,9S)-Benzyl 2-((2-aminoethyl)amino)-3-(benzyloxy)-1-bromo-6,7,8,8a,9,10-hexahydro-5H-9,4-b-(epiminoethano)phenanthrene-11-carboxylate (32)

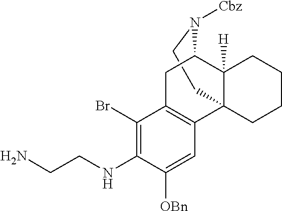 [SMILES: NCCNc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 3-(benzyloxy)-1-bromo-2-((2-(1,3-dioxoisoindolin-2-yl)ethyl)amino)-6,7,8,8a,9,10-hexahydro-5H-9,4-b-(epiminoethano)phenanthrene-11-carboxylate (31) (490 mg, 0.670 mmol) in MeOH (15 mL) was added hydrazine hydrate (0.16 mL, 3.35 mmol). The reaction mixture was stirred at r.t. overnight and evaporated under vacuum. The residue was purified by flash column chromatography (Biotage SP1™) to provide the title compound (430 mg, 90%) as a white solid.

MH+604.

### Step 3

### (6S,6aS,10aS)-Benzyl 12-(benzyloxy)-2,3,4,5,6,6a,7,8,9,10-decahydro-1H-6,10a-(epiminoethano)naphtho[2,1-f]quinoxaline-15-carboxylate (33)

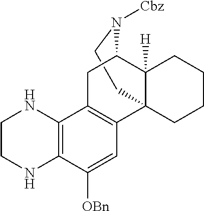 [SMILES: O=C(OCc1ccccc1)N1CC[C@@]23CCCC[C@@H]2[C@@H]1Cc1c3cc(OCc2ccccc2)c2c1NCCN2]

(4bS,8aS,9S)-Benzyl 2-((2-aminoethyl)amino)-3-(benzyloxy)-1-bromo-6,7,8,8a,9,10-hexahydro-5H-9,4-b-(epiminoethano)phenanthrene-11-carboxylate (32) (38 mg, 0.0630 mmol) was added to a microwave reactor containing a mixture of Pd₂(dba)₃ (5.8 mg, 0.00630 mmol), BINAP (5.9 mg, 0.00945 mmol) and sodium t-butoxide (17.0 mg, 0.176 mmol) in THF (5 mL). The capped reactor was placed in a microwave reactor and the mixture was irradiated at 170° C. for 25 min. The reaction mixture was evaporated to remove the solvent under vacuum. The residue was poured into water (20 mL) and extracted with EtOAc (20 mL×2). The organic phase was dried over MgSO₄ and evaporated under vacuum. The residue was purified by flash column chromatography (Biotage SP1™) to provide the title compound (27 mg, 81%) as a yellow solid.

¹H NMR (400 MHz, CDCl₃) δ 7.44-7.29 (m, 10H), 6.29 (s, 1H), 5.16-5.11 (m, 2H), 5.01 (s, 2H), 4.41 (d, J=41.2 Hz, 1H), 3.91-3.80 (m, 1H), 3.50 (br s, 2H), 3.39 (br s, 2H), 2.77-2.58 (m, 2H), 2.29-2.20 (m, 2H), 1.66-1.04 (m, 9H), 0.89-0.83 (m, 1H).

MH+524.

### Step 4

### (6S,6aS,10aS)-6,6a,7,8,9,10-Hexahydro-5H-6,10a-(epiminoethano)naphtho[2,1-f]quinoxalin-12-ol TFA salt (34)

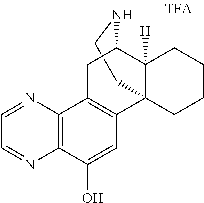 [SMILES: Oc1cc2c(c3nccnc13)C[C@@H]1NCC[C@]23CCCC[C@H]13]

To a solution of (6S,6aS,10aS)-benzyl 12-(benzyloxy)-2,3,4,5,6,6a,7,8,9,10-decahydro-1H-6,10a-(epiminoethano)naphtho[2,1-f]quinoxaline-15-carboxylate (33) (140 mg, 0.269 mmol) in IPA (10 mL) was added 10% Pd on charcoal (14 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (12 mg, 11%) as a yellow solid.

¹H NMR (400 MHz, CD₃OD) δ 8.82 (d, J=2.0 Hz, 1H), 8.73 (d, J=1.6 Hz, 1H), 7.15 (s, 1H), 3.80 (q, J=3.6 Hz, 1H), 3.48 (d, J=3.6 Hz, 2H), 3.02 (dd, J=9.2, 3.2 Hz, 1H), 2.64 (td, J=13.2, 4.0 Hz, 1H), 2.49 (d, J=14.0 Hz, 1H), 1.95 (dt, J=12.8, 3.2 Hz, 1H), 1.80 (td, J=14.4, 4.8 Hz, 1H), 1.67-1.23 (m, 6H), 1.21-1.06 (m, 2H).

MH+296.

### Example 10

### (4aS,14S,14aS)-2,3,4,13,14,14a-Hexahydro-1H-14,4a-(epiminoethano)naphtho[2,1-a]phenazin-6-ol TFA salt (40)

### Step 1

### (4bS,8aS,9S)-Benzyl 3-(benzyloxy)-2-((2-nitrophenyl)amino)-6,7,8,8a,9,10-hexahydro-5H-9,4-b-(epiminoethano)phenanthrene-11-carboxylate (36)

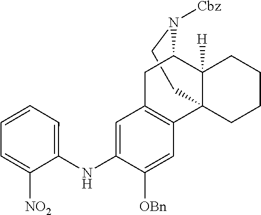 [SMILES: O=C(OCc1ccccc1)N1CC[C@@]23CCCC[C@@H]2[C@@H]1Cc1cc(Nc2ccccc2[N+](=O)[O-])c(OCc2ccccc2)cc13]

A mixture of (4bS,8aS,9S)-benzyl 2-amino-3-(benzyloxy)-6,7,8,8a,9,10-hexahydro-5H-9,4-b-(epiminoethano)phenanthrene-11-carboxylate (25) (2.5 g, 5.18 mmol), 1-chloro-2-nitrobenzene (1.63 g, 10.4 mmol), Pd(OAc)₂ (350 mg, 0.518 mmol), BINAP (650 mg, 1.04 mmol) and sodium t-butoxide (1.00 g, 10.4 mmol) in toluene (15 mL) was heated at 110° C. overnight and evaporated under vacuum. The residue was purified by flash column chromatography (Biotage SP1™) to provide the title compound (2.4 g, 77%) as a yellow solid.

MH+604.

### Step 2

### (4bS,8aS,9S)-Benzyl 3-(benzyloxy)-1-bromo-2-((2-nitrophenyl)amino)-6,7,8,8a,9,10-hexahydro-5H-9,4-b-(epiminoethano)phenanthrene-11-carboxylate (37)

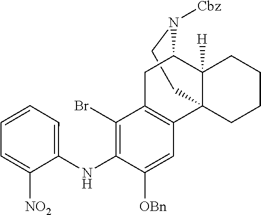 [SMILES: O=C(OCc1ccccc1)N1CC[C@@]23CCCC[C@@H]2[C@@H]1Cc1c3cc(OCc2ccccc2)c(Nc2ccccc2[N+](=O)[O-])c1Br]

A mixture of (4bS,8aS,9S)-benzyl 3-(benzyloxy)-2-((2-nitrophenyl)amino)-6,7,8,8a,9,10-hexahydro-5H-9,4-b-(epiminoethano)phenanthrene-11-carboxylate (36) (1.05 g, 1.74 mmol) and pyridinium tribromide (1.11 g, 3.48 mmol) in THF (20 mL) was heated at 60° C. overnight and evaporated under vacuum. The residue was further purified by flash column chromatography (Biotage SP1™) to provide the title compound (0.99 g, 83%) as a red solid.

MH+682.

### Step 3

### (4bS,8aS,9S)-Benzyl 2-((2-aminophenyl)amino)-3-(benzyloxy)-1-bromo-6,7,8,8a,9,10-hexahydro-5H-9,4-b-(epiminoethano)phenanthrene-11-carboxylate (38)

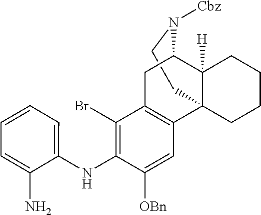 [SMILES: Nc1ccccc1Nc1c(OCc2ccccc2)cc2c(c1Br)C[C@H]1[C@H]3CCCC[C@@]23CCN1C(=O)OCc1ccccc1]

To a solution of (4bS,8aS,9S)-benzyl 3-(benzyloxy)-1-bromo-2-((2-nitrophenyl)amino)-6,7,8,8a,9,10-hexahydro-5H-9,4-b-(epiminoethano)phenanthrene-11-carboxylate (37) (410 mg, 0.601 mmol) and hydrazine hydrate (0.15 mL, 3.00 mmol) in MeOH (200 mL) was added Raney Ni (water solution, 1 mL) dropwise. The reaction mixture was stirred at r.t. for 2 hr. and filtered to remove the catalyst. The filtrate was evaporated under vacuum. The residue was further purified by flash column chromatography (Biotage SP1™) to provide the title compound (325 mg, 83%) as a yellow solid.

MH+652.

### Step 4

### (4aS,14S,14aS)-Benzyl 6-(benzyloxy)-2,3,4,13,14,14a-hexahydro-1H-14,4a-(epiminoethano)naphtho[2,1-a]phenazine-15-carboxylate (39)

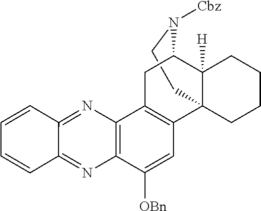 [SMILES: O=C(OCc1ccccc1)N1CC[C@@]23CCCC[C@@H]2[C@@H]1Cc1c3cc(OCc2ccccc2)c2nc3ccccc3nc12]

(4bS,8aS,9S)-Benzyl 2-((2-aminophenyl)amino)-3-(benzyloxy)-1-bromo-6,7,8,8a,9,10-hexahydro-5H-9,4-b-(epiminoethano)phenanthrene-11-carboxylate (38) (47 mg, 0.0720 mmol) was added to a microwave reactor containing a mixture of Pd₂(dba)₃ (6.6 mg, 0.00720 mmol), BINAP (6.7 mg, 0.0108 mmol) and sodium t-butoxide (13.8 mg, 0.144 mmol) in THF (5 mL). The capped reactor was placed in a microwave reactor and the mixture was irradiated at 170° C. for 40 min. The reaction mixture was evaporated to remove the solvent under vacuum. The residue was poured into water (20 mL) and extracted with EtOAc (20 mL×2). The organic phase was dried over MgSO₄ and evaporated under vacuum. The residue was purified by flash column chromatography (Biotage SP1™) to provide the title compound (19 mg, 47%) as a brown solid.

¹H NMR (400 MHz, CDCl₃) δ 8.42 (d, J=7.2 Hz, 1H), 8.22 (d, J=10.0 Hz, 1H), 7.85-7.80 (m, 1H), 7.53 (d, J=7.6 Hz, 1H), 7.43-7.26 (m, 10H), 6.97 (s, 1H), 5.62 (d, J=13.2 Hz, 1H), 5.47 (d, J=12.8 Hz, 1H), 5.20-5.08 (m, 2H), 4.60 (d, J=44.8 Hz, 1H), 3.97-3.85 (m, 1H), 3.51-3.48 (m, 2H), 2.71-2.59 (m, 1H), 2.21-2.17 (m, 1H), 1.82-1.21 (m, 8H), 0.88-0.82 (m, 2H).

MH+570.

### Step 5

### (4aS,14S,14aS)-2,3,4,13,14,14a-Hexahydro-1H-14,4a-(epiminoethano)naphtho[2,1-a]phenazin-6-ol TFA salt (40)

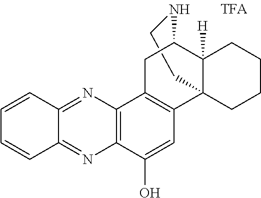 [SMILES: Oc1cc2c(c3nc4ccccc4nc13)C[C@@H]1NCC[C@]23CCCC[C@H]13]

To a solution of (4aS,14S,14aS)-Benzyl 6-(benzyloxy)-2,3,4,13,14,14a-hexahydro-1H-14,4a-(epiminoethano)naphtho[2,1-a]phenazine-15-carboxylate (39) (120 mg, 0.211 mmol) in IPA (5 mL) was added 10% Pd on charcoal (24 mg). The mixture was stirred under hydrogen atmosphere at r.t. overnight. The reaction mixture was filtered to remove the catalyst and evaporated under vacuum. The residue was further purified by prep. HPLC (Gilson, C18 column, 0.1% TFA) to provide the title compound (27 mg, 28%) as a brown solid.

¹H NMR (400 MHz, CD₃OD) δ 8.33-8.30 (m, 1H), 8.25-8.23 (m, 1H), 7.92-7.89 (m, 2H), 7.24 (s, 1H), 3.93 (d, J=3.6 Hz, 1H), 3.71 (d, J=3.2 Hz, 2H), 3.13 (dd, J=13.2, 3.2 Hz, 1H), 2.80 (td, J=13.2, 4.0 Hz, 1H), 2.61 (d, J=14.0 Hz, 1H), 2.07 (dt, J=12.4, 3.2 Hz, 1H), 1.90 (td, J=14.4, 4.8 Hz, 1H), 1.82-1.45 (m, 6H), 1.31-1.23 (m, 2H).

MH+346.

### Example 11

### (4aS,13S,13aS)-1,2,3,4,7,12,13,13a-Octahydro-13,4a-(epiminoethano)naphtho[1,2-c]carbazole-6,8-diol TFA salt (45)

### Step 1

### (4bS,8aS,9S)-Benzyl 3-hydroxy-2-iodo-6,7,8,8a,9,10-hexahydro-5H-9,4-b-(epiminoethano)phenanthrene-11-carboxylate (41)

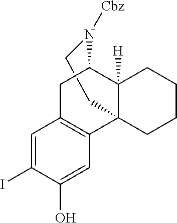 [SMILES: O=C(OCc1ccccc1)N1CC[C@@]23CCCC[C@@H]2[C@@H]1Cc1cc(I)c(O)cc13]

A mixture of (4bS,8aS,9S)-benzyl 3-hydroxy-6,7,8,8a,9,10-hexahydro-5H-9,4-b-(epiminoethano)phenanthrene-11-carboxylate (2) (10.0 g, 26.5 mmol) and iodine (13.4 g, 53.0 mmol) in pyridine (60 mL) was heated at 60° C. overnight and evaporated under vacuum. The residue was further purified by flash column chromatography (Biotage SP1™) to provide the title compound (12.9 g, 97%) as a yellow solid.

MH+504.

### Step 2

### (4bS,8aS,9S)-Benzyl 2-iodo-3-methoxy-6,7,8,8a,9,10-hexahydro-5H-9,4-b-(epiminoethano)phenanthrene-11-carboxylate (42)

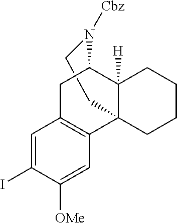 [SMILES: COc1cc2c(cc1I)C[C@H]1[C@H]3CCCC[C@@]23CCN1C(=O)OCc1ccccc1]

To a solution of (4bS,8aS,9S)-Benzyl 3-hydroxy-2-iodo-6,7,8,8a,9,10-hexahydro-5H-9,4-b-(epiminoethano)phenanthrene-11-carboxylate (41) (12.9 g, 25.6 mmol) and K₂CO₃ (5.31 g, 38.4 mmol) in acetone (100 mL) was added iodomethane (2.4 mL, 38.4 mmol). The reaction mixture was heated at 60° C. overnight and evaporated to remove the solvent under vacuum. The residue was poured into water (300 mL) and extracted with EtOAc (150 mL×3). The organic phase was dried over MgSO₄ and evaporated under vacuum. The residue was purified by flash column chromatography (Biotage SP1™) to provide the title compound (12.2 g, 92%) as a white solid.

MH+518.

### Step 3

### (4bS,8aS,9S)-Benzyl 3-methoxy-2-((2-methoxyphenyl)amino)-6,7,8,8a,9,10-hexahydro-5H-9,4-b-(epiminoethano)phenanthrene-11-carboxylate (43)

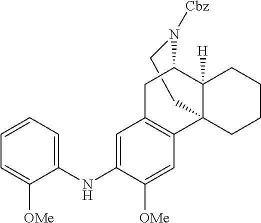 [SMILES: COc1ccccc1Nc1cc2c(cc1OC)[C@]13CCCC[C@@H]1[C@H](C2)N(C(=O)OCc1ccccc1)CC3]

A mixture of (4bS,8aS,9S)-Benzyl 2-iodo-3-methoxy-6,7,8,8a,9,10-hexahydro-5H-9,4-b-(epiminoethano)phenanthrene-11-carboxylate (42) (1.5 g, 2.90 mmol), o-anisidine (0.39 mL, 3.48 mmol), (dppf)PdCl₂.CH₂Cl₂ (94.7 mg, 0.116 mmol), dppf (175 mg, 0.348 mmol) and sodium t-butoxide (418 mg, 4.35 mmol) in toluene (15 mL) was heated at 100° C. overnight and evaporated under vacuum. The residue was purified by flash column chromatography (Biotage SP1™) to provide the title compound (1.42 g, 96%) as a yellow solid.

MH+513.

### Step 4

### (4aS,13S,13aS)-Benzyl 6,8-dimethoxy-1,2,3,4,7,12,13,13a-octahydro-13,4a-(epiminoethano)naphtho[1,2-c]carbazole-14-carboxylate (44)

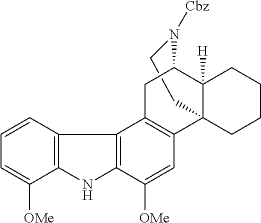 [SMILES: COc1cccc2c1[nH]c1c(OC)cc3c(c12)C[C@H]1[C@H]2CCCC[C@@]32CCN1C(=O)OCc1ccccc1]

A mixture of (4bS,8aS,9S)-benzyl 3-methoxy-2-((2-methoxyphenyl)amino)-6,7,8,8a,9,10-hexahydro-5H-9,4-b-(epiminoethano)phenanthrene-11-carboxylate (43) (513 g, 1.00 mmol), Pd(OAc)₂ (202 mg, 0.300 mmol) and Cu(OAc)₂ (272 mg, 1.50 mmol) in glacial acetic acid (10 mL) was heated at 110° C. for 2 days and evaporated under vacuum. The residue was purified by flash column chromatography (Biotage SP1™) to provide the title compound (312 mg, 61%) as a yellow solid.

¹H NMR (400 MHz, CDCl₃) δ 8.51 (s, 1H), 7.71 (dd, J=32.4, 4.0 Hz, 1H), 7.44-7.25 (m, 4H), 7.14 (t, J=8.0 Hz, 1H), 6.89 (s, 1H), 6.89 (d, J=7.6 Hz, 1H), 6.82 (s, 1H), 5.24-5.09 (m, 2H), 4.59 (d, J=44.8 Hz, 1H), 4.01 (s, 3H), 3.98 (s, 3H), 3.76-3.73 (m, 1H), 3.63-3.52 (m, 1H), 3.31 (t, J=17.2 Hz, 1H), 2.74-2.62 (m, 1H), 2.46 (d, J=12.0 Hz, 1H), 1.87-1.79 (m, 2H), 1.66-1.22 (m, 7H), 0.88-0.86 (m, 1H).

MH+511.

### Step 5

### (4aS,13S,13aS)-1,2,3,4,7,12,13,13a-Octahydro-13,4a-(epiminoethano)naphtho[1,2-c]carbazole-6,8-diol TFA salt (45)

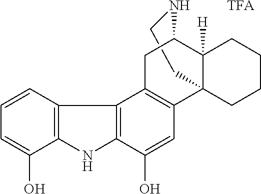 [SMILES: Oc1cccc2c1[nH]c1c(O)cc3c(c12)C[C@@H]1NCC[C@]32CCCC[C@H]12 ; "TFA"]

To a solution of (4aS,13S,13aS)-benzyl 6,8-dimethoxy-1,2,3,4,7,12,13,13a-octahydro-13,4a-(epiminoethano)naphtho[1,2-c]carbazole-14-carboxylate (44) (84 mg, 0.165 mmol) in DCM (10 mL) was added BBr₃ (1M in DCM, 0.83 mL, 0.830 mmol) at 0° C. The mixture was stirred at r.t. overnight and quenched by MeOH (5 mL). The reaction mixture was evaporated under vacuum. The residue was further purified by prep. HPLC (Waters, C18 column, 0.1% TFA) to provide the title compound (27 mg, 35%) as a brown solid.

¹H NMR (400 MHz, CD₃OD) δ 7.59 (d, J=8.0 Hz, 1H), 6.99 (t, J=8.0 Hz, 1H), 6.84-6.81 (m, 2H), 3.86-3.84 (m, 1H), 3.70-3.56 (m, 2H), 3.47 (d, J=19.2 Hz, 1H), 3.06 (dd, J=13.2, 3.2 Hz, 1H), 2.80 (td, J=13.2, 4.0 Hz, 1H), 2.50 (d, J=13.6 Hz, 1H), 1.97 (dt, J=12.8, 3.6 Hz, 1H), 1.82 (td, J=13.6, 4.8 Hz, 1H), 1.70-1.22 (m, 8H).

MH+349.

The following compounds of Example 12, 13 and 14 were obtained by repeating the procedure of Example 11.

### Example 12

### (4aS,13S,13aS)-1,2,3,4,7,12,13,13a-Octahydro-13,4a-(epiminoethano)naphtho[1,2-c]carbazol-6-ol TFA salt (45a)

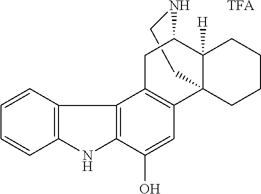 [SMILES: Oc1cc2c(c3c1[nH]c1ccccc13)C[C@@H]1NCC[C@]23CCCC[C@H]13 ; "TFA"]

¹H NMR (400 MHz, CD₃OD) δ 8.08 (d, J=8.0 Hz, 1H), 7.57 (d, J=8.0 Hz, 1H), 7.35 (t, J=8.0 Hz, 1H), 7.15 (t, J=8.0 Hz, 1H), 6.86 (s, 1H), 3.88-3.87 (m, 1H), 3.71 (dd, J=18.8, 6.4 Hz, 1H), 3.49 (d, J=18.8 Hz, 1H), 3.07 (dd, J=13.2, 3.6 Hz, 1H), 2.80 (td, J=13.2, 3.6 Hz, 1H), 2.51 (d, J=14.0 Hz, 1H), 1.99 (d, J=6.4 Hz, 1H), 1.83 (td, J=13.6, 4.8 Hz, 1H), 1.67-1.20 (m, 8H).

MH+333.

### Example 13

### (4aS,13S,13aS)-1,2,3,4,7,12,13,13a-Octahydro-13,4a-(epiminoethano)naphtho[1,2-c]carbazole-6,10-diol TFA salt (45b)

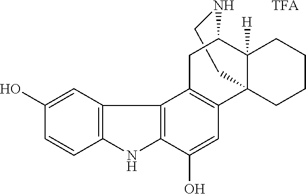 [SMILES: Oc1ccc2[nH]c3c(O)cc4c(c3c2c1)C[C@@H]1NCC[C@]42CCCC[C@H]12 ; "TFA"]

¹H NMR (400 MHz, CD₃OD) δ 7.49 (d, J=2.0 Hz, 1H), 7.34 (d, J=8.8 Hz, 1H), 6.93 (dd, J=8.8, 2.4 Hz, 1H), 6.81 (s, 1H), 3.87-3.85 (m, 1H), 3.66 (dd, J=18.8, 6.4 Hz, 1H), 3.44 (d, J=19.2 Hz, 1H), 3.07 (dd, J=12.8, 3.2 Hz, 1H), 2.80 (td, J=13.6, 3.6 Hz, 1H), 2.49 (d, J=13.2 Hz, 1H), 1.97 (d, J=12.4 Hz, 1H), 1.82 (td, J=13.6, 4.8 Hz, 1H), 1.67-1.24 (m, 8H).

MH+349.

### Example 14

### (4aS,13S,13aS)-10-Methyl-1,2,3,4,7,12,13,13a-octahydro-13,4a-(epiminoethano)naphtho[1,2-c]carbazol-6-ol TFA salt (45c)

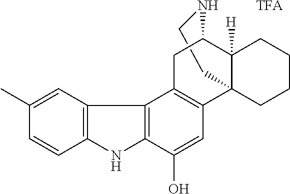 [SMILES: Cc1ccc2[nH]c3c(O)cc4c(c3c2c1)C[C@@H]1NCC[C@]42CCCC[C@H]12 ; "TFA"]

¹H NMR (400 MHz, CD₃OD) δ 7.88 (s, 1H), 7.40 (d, J=8.0 Hz, 1H), 7.20 (dd, J=8.4, 1.2 Hz, 1H), 6.84 (s, 1H), 3.88-3.86 (m, 1H), 3.70 (dd, J=18.8, 6.4 Hz, 1H), 3.49 (d, J=18.8 Hz, 1H), 3.07 (dd, J=13.2, 3.2 Hz, 1H), 2.80 (td, J=13.2, 3.6 Hz, 1H), 2.52-2.50 (m, 4H), 1.99 (d, J=12.0 Hz, 1H), 1.83 (td, J=13.6, 4.8 Hz, 1H), 1.71-1.22 (m, 8H).

MH+347.

### Experiment 1: Cell Cytotoxicity Test

HT22 cells (mouse hippocampal neuron, Salk Institute and KRIBB) were placed in a 96-well plate 3×10³ cells/well for 16 h before treatment. Five millimolar glutamate and the inventive compounds were co-treated and incubated for 24 h in growth media (DMEM with 10% FBS and 1% penicillin streptomycin). MTT (3-(4,5-dimethylthazol-2-yl)-2,5-diphenyltetrazolium bromide, Sigma®) was treated for 4 h and detected with a plate reader at a wavelength=450 nm [Da-Qing, et. al., Anti-oxidant and anti-inflammatory activities of macelignan in murine hippocampal cell line and primary culture of rat microglia cells, BBRC, 2005, 331, 1264-1269].

The EC₅₀ values were statistically analyzed using Prism® (GraphPad Software Inc., San Diego, Calif.). The results of the cell cytotoxicity test are summarized in Table 1 (EC₅₀: neuroprotective effect against glutamate toxicity, and CC₅₀: cytotoxicity of compound).

TABLE 1

| | | |
|---|---|---|
| Example No. | EC₅₀ | CC₅₀ |

| | | | | |
|---|---|---|---|---|
| Example 1 | 26.9 | μM | >100 | μM |
| Example 2 | 27.7 | μM | >100 | μM |
| Example 3 | 8.5 | μM | >100 | μM |
| Example 4 | 9.6 | μM | >100 | μM |
| Example 5 | 4.97 | μM | >100 | μM |
| Example 6 | 314 | nM | >100 | μM |
| Example 7 | 650 | nM | 13.5 | μM |
| Example 8 | 457 | nM | 8.1 | μM |
| Example 9 | 16.3 | μM | 44.8 | μM |
| Example 10 | 354 | nM | 10.7 | μM |
| Example 11 | 303 | nM | 44 | μM |
| Example 12 | 710 | nM | 49.8 | μM |
| Example 13 | 1.11 | μM | >100 | μM |
| Example 14 | 908 | nM | 5.7 | μM |

As shown in Table 1, the inventive compounds obtained in Examples 1 to 14 are effective as a neuroprotective agent.

What is claimed is:

1. A compound of formula (I), or a prodrug or a pharmaceutically acceptable salt thereof: 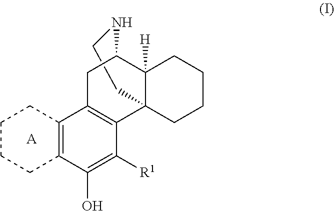
wherein,
R¹ is H, C₁₋₃ alkyl, C₁₋₂ alkoxy, or halogen; and
ring A is a saturated or unsaturated 5 to 9-membered heteromonocyclic ring, or a saturated or unsaturated fused 9 to 16-membered heterobicyclic ring,
said heteromonocyclic and heterobicyclic rings each independently contains at least one heteroatom selected from N and O, and
said ring A is optionally substituted by at least one substituent selected from the group consisting of halogen, hydroxy, C₁₋₄ alkyl, C₁₋₄ alkoxy, C₆₋₁₂ aryl, and C₃₋₁₁ heteroaryl.

2. The compound according to claim 1, which is represented by formula (Ia): 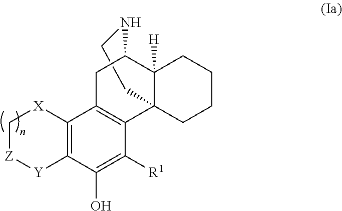
wherein,
n is 1 or 2;
R¹ is H, halogen, C₁₋₃ alkyl, or C₁₋₂ alkoxy;
X and Y are each independently —NH—, —N(R²)—, —N(C(═O)R²)— or —O—;
Z is —CH₂— or —CH(R²)—;
Y and Z are optionally connected together to form 5 to 9-membered carbocyclic or heterocyclic ring; and
R² is H, C₁₋₄ alkyl, C₆₋₁₂ aryl, or C₃₋₁₁ heteroaryl.

3. The compound according to claim 1, which is represented by formula (Ia′): 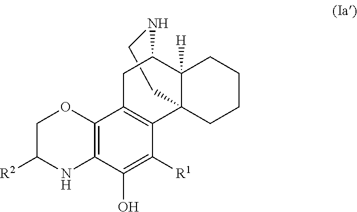
wherein,
R¹ is H, halogen, C₁₋₃ alkyl, or C₁₋₂ alkoxy; and
R² is H, C₁₋₄ alkyl, C₆₋₁₂ aryl, or C₃₋₁₁ heteroaryl.

4. The compound according to claim 1, which is represented by formula (Ib): 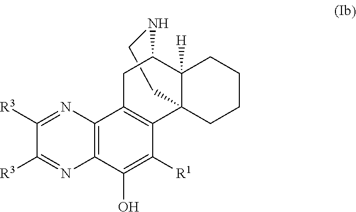
wherein,
R¹ is H, halogen, C₁₋₃ alkyl, or C₁₋₂ alkoxy; and
R³ is H, C₁₋₄ alkyl, C₆₋₁₂ aryl, or C₃₋₁₁ heteroaryl.

5. The compound according to claim 1, which is represented by formula (Ib′): 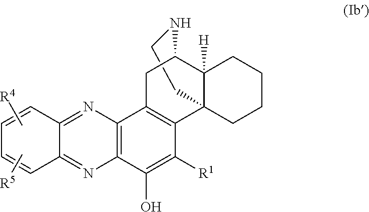
wherein,
R¹ is H, halogen, C₁₋₃ alkyl, or C₁₋₂ alkoxy; and
R⁴ and R⁵ are each independently H, C₁₋₄ alkyl, C₆₋₁₂ aryl, or C₃₋₁₁ heteroaryl.

6. The compound according to claim 1, which is represented by formula (Ic): 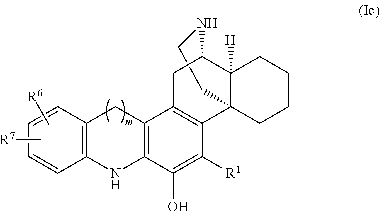
wherein,
m is an integer ranging from 0 to 3;
R¹ is H, halogen, C₁₋₃ alkyl, or C₁₋₂ alkoxy; and
R⁶ and R⁷ are each independently H, halogen, hydroxy, C₁₋₄ alkyl, C₁₋₄ alkoxy, C₆₋₁₂ aryl, or C₃₋₁₁ heteroaryl.

7. The compound according to claim 1, which is selected from the group consisting of:
(6aS,10aS)-2,3,4,5,6,6a,7,8,9,10-Decahydro-6,10a-(epiminoethano)phenanthro[2,1-b][1,4]oxazin-12-ol trifluoroacetic acid (TFA) salt;
(7S,7aS,11aS)-3,4,5,6,7,7a,8,9,10,11-Decahydro-2H-7,11a-(epiminoethano)phenanthro[2,1-b][1,4]oxazepin-13-ol TFA salt;
1-((6S,6aS,10aS)-12-Hydroxy-2,3,6,6a,7,8,9,10-octahydro-6,10a-(epiminoethano)phenanthro[2,1-b][1,4]oxazin-4(5H)-yl)ethanone TFA salt;
(6aS,10aS)-4-Methyl-2,3,4,5,6,6a,7,8,9,10-decahydro-6,10a-(epiminoethano)phenanthro[2,1-b][1,4]oxazin-12-ol TFA salt;
(6aS,10aS)-3,5,6,6a,7,8,9,10-Octahydro-2H-6,10a-(epiminoethano)phenanthro[1,2-b][1,4]dioxin-12-ol TFA salt;
(6aS,10aS)-2-Methyl-1,2,3,5,6,6a,7,8,9,10-decahydro-6,10a-(epiminoethano)phenanthro[1,2-b][1,4]oxazin-12-ol TFA salt;
(6aS,10aS)-1,2,3,5,6,6a,7,8,9,10-Decahydro-6,10a-(epiminoethano)phenanthro[1,2-b][1,4]oxazin-12-ol TFA salt;
6aS,10aS)-2-Phenyl-1,2,3,5,6,6a,7,8,9,10-decahydro-6,10a-(epiminoethano)phenanthro[1,2-b][1,4]oxazin-12-ol TFA salt;
6aS,10aS)-6,6a,7,8,9,10-Hexahydro-5H-6,10a-(epiminoethano)naphtho[2,1-f]quinoxalin-12-ol TFA salt;
(4aS,14S,14aS)-2,3,4,13,14,14a-Hexahydro-1H-14,4a-(epiminoethano)naphtho[2,1-a]phenazin-6-ol TFA salt;
(4aS,13S,13aS)-1,2,3,4,7,12,13,13a-Octahydro-13,4a-(epiminoethano)naphtho[1,2-c]carbazole-6,8-diol TFA salt;
(4aS,13S,13aS)-1,2,3,4,7,12,13,13a-Octahydro-13,4a-(epiminoethano)naphtho[1,2-c]carbazol-6-ol TFA salt;
(4aS,13S,13aS)-1,2,3,4,7,12,13,13a-Octahydro-13,4a-(epiminoethano)naphtho[1,2-c]carbazole-6,10-diol TFA salt; and
(4aS,13S,13aS)-10-Methyl-1,2,3,4,7,12,13,13a-octahydro-13,4a-(epiminoethano)naphtho[1,2-c]carbazol-6-ol TFA salt.

8. A pharmaceutical composition for treating or preventing a neurodegenerative disease, comprising the compound according to claim 1 and a pharmaceutically acceptable carrier.

9. The pharmaceutical composition of claim 8, wherein the neurodegenerative disease is selected from the group consisting of Alzheimer's disease (AD), Parkinson's disease (PD), Huntington's disease (HD), amyotrophic lateral sclerosis (ALS), and ischemic stroke.

10. A method for treating or preventing a neurodegenerative disease, comprising administering to a patient in need of treatment thereof the compound according to claim 1.

11. A use of the compound according to claim 1 for the manufacture of a medicament for preventing or treating a neurodegenerative disease.
